SEQUENCE LISTING (3 sequences)

SEQ ID NO: 1 - DNA, 24 bp, Artificial Sequence
  synthetic. PCR probe
tatgagtgtcgtacagcctccagg

SEQ ID NO: 2 - DNA, 20 bp, Artificial Sequence
  synthetic. forward primer
cttcacgcagaaagcgtcta

SEQ ID NO: 3 - DNA, 20 bp, Artificial Sequence
  synthetic. reverse primer.
caagcaccctatcaggcagt

(19) United States
(12) Patent Application Publication
(10) Pub. No.: US 2011/0064694 A1
(43) Pub. Date: Mar. 17, 2011

(54) ANTI-HEPATITIS C ACTIVITY OF MESO-TETRAKIS-PORPHYRIN ANALOGUES

(75) Inventors: Andrew HAMILTON, Oxford (GB); Yung-Chi CHENG, Woodbridge, CT (US)

(73) Assignee: YALE UNIVERSITY, New Haven, CT (US)

(21) Appl. No.: 12/877,544

(22) Filed: Sep. 8, 2010

Related U.S. Application Data
(60) Provisional application No. 61/276,273, filed on Sep. 9, 2009.

(51) Int. Cl. A61K 38/21; C07D 487/22; A61K 31/41; A61K 31/555; A61K 31/7056; A61K 33/24; A61P 31/12; A61P 35/00

(52) U.S. Cl. 424/85.4; 540/145; 514/359; 514/185; 514/43; 424/649

(57) ABSTRACT

The present invention relates to porphyrin analogues, their use in pharmaceutical compositions alone, or combination with other agents and in the treatment and/or prophylaxis of flaviviridae viral infections, especially hepatitis C viral infection, and secondary disease states and/or conditions associated with same.

### RELATED APPLICATIONS

This application claims priority from provisional application Ser. No. 61/276,273, filed Sep. 9, 2009 entitled Anti-hepatitis C Activity of meso-tetrakis-porphyrin analogues, the entire contents of which application is incorporated by reference herein.

### GRANT SUPPORT

This invention was made with government support under NIH AI-073299 awarded by the National Institutes of Health. The government has certain rights in the invention.

### FIELD OF THE INVENTION

The present invention relates to porphyrin analogues, their use in pharmaceutical compositions and in the treatment and/or prophylaxis of hepatitis C viral infections and related disease states and/or conditions.

### BACKGROUND OF THE INVENTION

Hepatitis C virus (HCV) exerts an increasingly heavy burden on global healthcare and approximately 200 million people worldwide are infected (39). Chronically infected patients are often at risk for developing hepatic fibrosis, cirrhosis, and hepatocellular carcinoma (15). HCV is an enveloped virus that belongs to the Flaviviridae family, and seven recognized HCV genotypes and numerous subtypes have been identified. Genotype 1a is the most prevalent strain worldwide and genotype 1b is predominant in Europe and North America, whereas genotypes 2 is more prevalent in Asia (4, 29). The current standard of care pegylated IFNcI combined with ribavirin is plagued with adverse effects and has sustained viral response in less than half of the patients with genotype 1 infections (11, 17,25). Therefore more effective and better tolerated therapies are urgently needed, in particular for the treatment of non-responders to IFN-based therapies.

The HCV genome, which is a single-stranded positive-sense RNA about 9.6-kb in length, encodes a polyprotein that is cleaved by viral and host proteases into structural (core, E1,82, and possibly p7) and nonstructual proteins (NS2, NS3, NS4A, NS4B, NS5A, and NS5B) (4). The nonstructural proteins NS3 through NS5B assemble on the cytoplasmic membranes into a well-organized macromolecular machinery called the HCV replicase that is essential for the viral RNA replication (8, 14, 32). Until recently, the development of anti-HCV drugs had been hindered by the lack of a robust cell culture model. The establishment and optimization of the replicon systems have extensively widened our knowledge of the HCV replication, and also proved a powerful tool for the discovery of novel agents that target the assembly and function of HCV replicase. HCV replicons are subgenomic constructs capable of autonomous replication in hepatoma cell lines, and the major viral components of the replicons consist of NS3 through NS5B (2, 23). Amongst these nonstructural proteins, viral protease NS3/44 and RNA-dependent RNA polymerase NS5B are the most extensively explored targets for anti-HCV drug development (for reviews, see Ref (6,24,28)). However, due to the error-prone nature of NS5B, mutational escapes could rapidly emerge under selective pressures from viral-specific inhibitors (35, 40). Other modalities under investigation include immune modulators and therapies targeting viral RNA.

Protein-protein interactions often involve substantial interfacial areas larger than 1000 Å² (34). Yet selective targeting of a surface region in order to alter a protein's function or interaction with other biomolecules has not been extensively explored. In the current study, the present inventors have designed and synthesized a class of theoretical protein-binding molecules built on a porphyrin core, which is compatible with the biological milieu. The tetraphenylporphyrin scaffold provides a sizable platform allowing hydrophobic interactions with the target surface, while charged peptidic appendages projected from the periphery support electrostatic interactions with complementary groups on the target(s). This contact with large area may decrease the likelihood of high resistance developing of targeted virus. Pursuant to the present invention, the inventors explored the antiviral potential of this class of compounds against the HCV replicon systems. Meso-teftakis-(3,5-dicarboxy-4,4′-biphenyl)porphyrin (compound 6) was found to be the most potent and selective inhibitor of HCV genotype 1b Conl replicons (EC₅₀ 0.024±0.005) with low cytotoxicity. While undertaking mechanistic studies to characterize the molecular target(s), the examples here describe the structure-activity relationships of tetraphenylporphyrin derivatives and the anti-HCV properties of compound 6, which is a proof-of-concept model for the development of proteomimetics in HCV drug discovery.

### OBJECTS OF THE INVENTION

It is an object of the present invention to provide novel compounds for the treatment and/or inhibition of HCV infections.

It is another object of the invention to provide pharmaceutical compositions for use in the treatment and/or inhibition of HCV infections.

It is an additional object of the invention to provide methods of treating and/or inhibiting HCV infections.

Any one or more of these and/or additional objects of the invention may be readily gleaned from a description of the invention which follows.

### BRIEF DESCRIPTION OF THE FIGURES

FIG. 1 shows representative chemical structures of the compounds used in this study. The tetraphenylporphyrin analogues compounds 1-3, and tetrabiphenylporphyrin analogues compounds 4-9 were shown with their synthetic intermediates. TPPS+ and TTMAPP were purchased from Sigma-Aldrich, and compound 1 from Frontier Scientific, Inc.

FIG. 2 shows the time- and dose-dependent reduction of viral parameters in genotype 1b (Conl) replicon cells induced by compound 6. Huh-luc/neo-ET cells were incubated with serially diluted compound 6 for 24, 48, or 72 hrs. Results were expressed as percentage of mocktreated controls. (A) shows the reduction of reporter luciferase activity, which indirectly reflects the replication level of HCV replicons. (B) shows the reduction of HCV RNA level normalized against the mRNA level of human B-actin. (C) shows the reduction of the NS5A protein level as in one experiment. (D) NS5A protein level quantitated and normalized against the protein level of o-tubulin (mean t S.D. from three experiments).

FIG. 3 shows the effect of serum binding on the 72-hr EC of compounds 1, 4 &. 6. The fold changes in EC₅₀ were plotted against percentage serum using the EC₅₀ at 5% FBS as one fold. The antiviral activities of compounds 1 and 6 decreased linearly with increasing serum, and also differed significantly from each other at the 95% confidence intervals. Change in the EC₅₀s of compound 4, which was the least affected by serum binding, was nonlinear. P value was determined by two-way ANOVA test using GraphPad Prism 4.0.

FIG. 4 shows that compound 6 could prevent the rebound of genotype 1b (Conl) replicons. Replicon cells were incubated with increasing concentrations of compound 6 for twelve days free from G418. At the end of incubation, compound 6 was removed and 250 μg/ml G418 was reintroduced. (A) The level of HCV RNA in cells was quantitated by qRT-PCR. RNA copy number per pg of total RNA was expressed as ratio relative to the mocktreated controls. During the rebound period, replicon cells incubated with 300 nM and 1 μM of compound 6 were not confluent enough for sampling. (B) Cell viability was shown on logro scale as percentage of mock-treated controls. Replicon cells that were treated with 300 nM and 1 μM of compound 6 were no longer viable by Day 21.

FIG. 5 shows that, compared with genotype 1b (Conl) replicons, genotype 2a (JFH-I) replicon was more resistant to both compound 6 and IFNu-2a. Genotype 1b (Conl) replicon cells Huh-luc/neo-ET and genotype 2a (JFH-1) replicon cells YSGR-JFH were incubated with increasing concentrations of (A) compound 6 or (B) IFNu-2a. Cells were harvested 72 hrs after incubation. The HCV RNA level was quantitated by q RT-PCR and expressed as percentage of mock-treated controls.

FIG. 6 shows an antiviral isobologram and CIqo plot of compound 6 in combination with BILN 2061 or IFNα-2a in vitro. Huh-luc/neo-ET cells were co-incubated for 72 hrs with various concentrations of Drug 1 or 2 alone or the two in combination at different potency ratios. (4, C) The ratios of the apparent EC₅₀ of each drug in combination over its EC₅₀ when applied alone were plotted against each other in isobolograms. The hypotenuse represents linear additive response to the action of two therapeutic agents. Isobols that bow below the hypotenuse indicate synergism, and isobols that bow above the hypotenuse indicate antagonism. Experimental data points on the isobol represents a combination that inhibits the HCV replication by 50% and hence isoeffective with the line of additivity. Synergy index (SD values were calculated as the fractional distance from the origin to the intersection of isobole and hypotenuse, with the total distance (half the length of hypotenuse) designated as value 1.00. Therefore the smaller the SI value the stronger the degree of synergism. The EC₅₀ isobolograms of compound 6 in combination with BILN 2061 or IFNα-2a were shown in (B) and (D) respectively (mean±S.D. from at least four independent experiments). Different degrees of synergism/antagonism are expected at different effect levels. The combination index (CI, which equals (D)₁/(Dₓ)₁+(D)₂/(Dₓ)₂) at 90% effect level for the combination of compound 6 with BILN 2061 or IFNα-2a were plotted in (E) and (F) respectively. Mean CI₉₀ value for each dose ratio was indicated above the bars. CI=1+0.1 suggests additivity as indicated by the dashed line. CI value below the boundary indicates synergism and above, antagonism.

FIG. 7 shows the dose-dependent reduction of viral parameters in genotype 1b (Conl) cell line 429/BBix 72 hrs after incubation with compound 6. (A) Reduction of HCV RNA level. Results were normalized against the mRNA level of human B-actin and expressed as percentage of mock-treated control. (B) Reduction of the NS5A protein level.

### BRIEF DESCRIPTION OF THE INVENTION

The present invention relates to compounds according to the chemical structure:

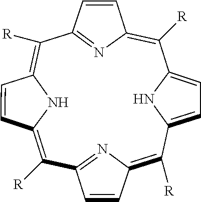

Where each R group is independently a substituted phenyl group, wherein said phenyl group is substituted with at least one (1, 2 or 3) carboxylic acid group(s) or at least one group (1, 2 or 3) containing a carboxylic acid group, including a group derived from an amino acid, or a biphenyl group which is substituted on the distil phenyl group (i.e., the phenyl group which is not attached to the porphyrin ring) with at least one (up to three) carboxylic acid group(s) or at least one (up to three) group(s) containing a carboxylic acid group, including a group derived from an amino acid, or a pharmaceutically acceptable salt, solvate or polymorph thereof, optionally in combination with a metal, with the proviso that when each R in said compound is identical and is a phenyl group substituted with only one group, that group is other than a carboxylic acid group.

In preferred aspects of the invention, all R groups in the compound are identical. Compounds according to the present invention may be optionally (metallated), i.e., they form a complex with a metal cation such as iron III (Fe3+), iron II, Cu II, Zn II, Mg II or another metal species, which may be neutral or charged. In particularly preferred compounds according to the present invention, R is a biphenyl group which is substituted with two carboxylic acid groups at the meta positions of the distil (furthest removed from the porphyrin ring) phenyl group (“di-metacarboxylic acid substituted bi-phenyl group”) or a pharmaceutically acceptable salt thereof.

In preferred aspects of the present invention, R is a —X—(CH₂)ₙCOOH group, a —X—(CH₂O)ⱼCOOH group, a —X—(CH₂CHYO)ₖCOOH group, a C(O)—NZ—(CH₂)ₘCOOH group, a

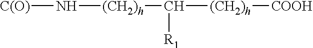

group, an optionally substituted biphenyl group wherein at least the distil phenyl contains at least one and up to three R′ group(s) (preferably two meta substituted carboxylic acid groups on the distill phenyl group), where R′ is a —X—(CH₂)ₙCOOH group, a —X—(CH₂O)ⱼCOOH group, a —X—(CH₂CHYO)ₖCOOH group, a C(O)—NZ—(CH₂)ₘCOOH group or a

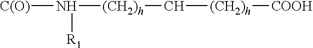

group;
R₁ is an amino acid sidechain (i.e., a sidechain normally bonded to the carbon atom and in the same manner as in an α-amino acid), preferably a sidechain derived from alanine, arginine, asparagine, aspartic acid, cysteine, glutamine, glutamic acid, glycine, histidine, isoleucine, leucine, lysine, methionine, norleucine, phenylalanine, proline (the carbon to which R₁ is attached and the alpha amine form a five membered heterocyclic ring), serine, threonine, tryptophan, tyrosine or valine (in cases where the amino acid sidechain is derived from aspartic acid or glutamic acid, one of the two carboxylic acid groups in the sidechain may be optionally esterified with a C₁-C₆ alkyl group), preferably R₁ is H, C₁-C₄ alkyl, CH₂OH, C₂-C₄ thioether (methionine), benzyl (phenylalanine) and p-hydroxybenzyl (tyrosine);
X is absent (i.e., a bond), O, S or N—Z;

### Y is H or CH₃;

Z is H or a C₁-C₃ alkyl group;
h is independently 0 to 2, preferably 0 or 1, more preferably 0;
j is an integer from 0 to 10, preferably 1 to 10;
k is an integer from 0 to 6, preferably 1 to 6;
m is an integer from 0 to 10, preferably 1 to 10;
n is an integer from 0 to 12, preferably 1 to 12; and
n′ is an integer from 0 to 12, preferably 1 to 12; or
a pharmaceutically acceptable salt, solvate or polymorph thereof.

Pharmaceutical compositions according to the present invention comprise an effective amount of at least one porphyrin compound as otherwise described above in combination with a pharmaceutically acceptable carrier, additive or excipient, said composition being adapted for administration to a patient or subject. Pharmaceutical compositions may also include an effective amount of a second anti-HCV agent including, for example interferon (IFN), including IFNα-2a and pegylated IFN, ribavirin or a combination of the two (REBETRON). Additional anti-HCV agents may include one or more of the following: BILN 2061, G418, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, VCH-222, TLR9 Agonist, PHX1766, SP-30 and mixtures thereof. Additional agents which may be included in pharmaceutical compositions according to the present invention include anti-cancer agents, among others. Preferred anti-cancer agents include liver anti-cancer agents, including doxorubicin, cis platin and mixtures thereof.

Methods of treating and/or inhibiting a Flaviviridea viral infection, especially HCV infections (including recurrent HCV infection and drug resistant HCV infection) in patients, especially human patients, represents a further aspect of the invention. In this method, an effective amount of a compound according to the present invention is administered to a patient or subject infected with Flaviviridea, especially an HCV infection in order to treat or inhibit the viral infection in the patient or subject.

Additional aspects of the present invention relate to methods for reducing the likelihood that a patient or subject at risk for a Flaviviridea infection, especially HCV infection (including drug resistant HCV), will contract the infection comprising administering an effective amount of at least one porphyrin compound as otherwise described above, in combination with a pharmaceutically acceptable carrier, additive or excipient, optionally in combination with at least one additional anti-HCV agent.

Another aspect of the present invention relates to methods for reducing the likelihood that a patient at risk for HCV relapse will relapse, said method comprising administering to a patient or subject at risk for HCV relapse an effective amount of at least one porphyrin compound as otherwise described herein, in combination with a pharmaceutically acceptable carrier, additive or excipient, optionally in combination with at least one additional anti-HCV agent.

Further aspects of the invention relate to methods for reducing the likelihood that a patient or subject will suffer a complication of an HCV infection (“secondary disease state or condition of HCV”), including cirrhosis of the liver, AIDS and/or cancer secondary to said infection, cryoglobulinemia (production of cryoglobulins), skin conditions (lichen planus and porphyria cutanea tarda), diabetes (primarily type II), platelet destruction (decrease in production of clotting factors), cancer (especially B cell lymphoma and/or hepatocellular cancer) and Raynaud's disease, said method comprising administering to a patient infected with HCV an effective amount of at least one porphyrin compound as otherwise described above, in combination with a pharmaceutically acceptable carrier, additive or excipient.

### DETAILED DESCRIPTION OF THE INVENTION

The following terms are used to describe the present invention. The definition of the terms is to be gleaned from a description of the invention and application of the term within the context of its use. In instances where a definition is not provided, the term shall be given the typical meaning understood by those of ordinary skill within the context of its use.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In the case of the numerical range of atoms which is used to describe a molecule, the numerical range shall be understood to include each and every positive integer within that range. By way of example, a C₁-C₆ alkyl group refers to an alkyl group which contains between one and six carbon atoms and all alkyl groups which individually contain 1, 2, 3, 4, 5 or 6 carbon atoms.

As discussed above, unless defined otherwise, all technical and scientific terms used herein have the same meaning as commonly understood by those of ordinary skill in the art to which this invention applies. Although any methods and materials similar or equivalent to those described herein can also be used in the practice or testing of the present invention, the preferred methods and materials are now described.

It is to be further noted that as used herein and in the appended claims, the singular forms “a,” “and” and “the” include plural references unless the context clearly dictates otherwise.

The term “patient” or “subject” is used throughout the specification to describe an animal, generally a mammal and preferably a human, to whom treatment, including prophylactic treatment, with the compositions according to the present invention is provided. For treatment of those infections, conditions or disease states which are specific for a specific animal such as a human patient, the term patient refers to that specific animal.

The term “effective” is used herein, unless otherwise indicated, to describe an amount of a compound or composition or component which, in context, is used to produce or effect an intended result, whether that result relates inter alia to the treatment of a viral, microbial or other disease state, especially an HCV infection, a disorder or condition associated with HCV as otherwise described herein or alternatively, is used to produce another compound, agent or composition. This term subsumes all other effective amount or effective concentration terms which are otherwise described in the present application.

The term “compound”, as used herein, unless otherwise indicated, refers to any specific chemical compound disclosed herein and includes tautomers, regioisomers, geometric isomers, and where applicable, optical isomers (enantiomers) thereof, as well as pharmaceutically acceptable salts thereof. Within its use in context, the term compound generally refers to a single compound, but also may include other compounds such as stereoisomers, regioisomers and/or optical isomers (including racemic mixtures) as well as specific enantiomers or enantiomerically enriched mixtures of disclosed compounds. The breadth of the term “compound” shall be construed within the context of the use of the term.

The term “Flaviviridae” refers to a family of viruses that infect mammals. The taxon includes the Flavivirus (yellow fever, West Nile virus), Pestivirus (classical swine fever or hog cholera), and Hepacivirus (Hepatitis C) groups. The genome of the Flaviviridae viruses is a monopartite, linear, single-stranded RNA of positive sense that is 10,000-11,000 nucleotides long. The 5′-terminus carries a methylated nucleotide cap or a genome-linked protein. The virus itself is enveloped and spherical, about 40-60 nm in diameter.

Flaviviridae viruses which are treated or otherwise impacted by compounds according to the present invention include Hepatitis C virus (HCV), bovine viral diarrhea virus (BVDV), hog cholera (swine fever), yellow fever and West Nile virus.

The term “Hepatitis C Virus” refers to a virus which causes an infection of the liver, which often becomes chronic and can lead to secondary disease states and/or conditions such as liver inflammation, AIDS and/or cancer. It is difficult for the human immune system to eliminate the virus from the body, and infection with HCV usually becomes chronic. Over a number of years and in some cases, decades, chronic infection with HCV damages the liver and can cause liver failure in some people. When the virus first enters the body, there usually are no symptoms, but over time, up to 85-90+% of newly infected people fail to clear the virus and become chronically infected. Currently, in the U.S., more than three million people are chronically infected with HCV. In the United States, there are 8,000 to 10,000 deaths each year related to HCV. HCV is the leading cause of liver transplantation in the U.S and is a risk factor for liver cancer. In Asia, HCV infection is a particularly problematic disease.

All hepatitis C viruses are made up of an outer coat (envelope) and contain enzymes and proteins that allow the virus to reproduce within the cells of the body, in particular, the cells of the liver. Although this basic structure is common to all hepatitis C viruses, there are at least six distinctly different strains of the virus which have different genetic profiles (genotypes). The present invention is directed to the treatment and/or prophylaxis of all strains of HCV which are susceptible to treatment. In the U.S., genotype 1 is the most common form of HCV. Even within a single genotype there may be some variations (genotype 1a and 1b, 2a and 2b, for example). Recognition of genotyping may be important to guide treatment, especially in the case of a cocktail of compounds which include the present compounds, because some viral genotypes respond better to certain types of therapy than others. Unless otherwise indicated, the term HCV shall refer to all six genotypes (1, 2, 3, 4, 5, and 6) and their subgenotypes.

The presence of HCV in the liver triggers biological processes which lead to inflammation. Over time (usually years, if not decades), prolonged inflammation may cause scarring. Extensive scarring in the liver is called cirrhosis. When the liver becomes cirrhotic, the liver fails to adequately perform its normal functions, (liver failure), and leads to serious complications and even death. Cirrhotic livers also are more prone to become cancerous.

About 75% of people have no symptoms when they first acquire HCV infection. The remaining 25% may complain of fatigue, loss of appetite, muscle aches or fever. Yellowing of the skin or eyes (jaundice) is rare at this early stage of infection. Over time, the liver in people with chronic infection may begin to experience the effects of the persistent inflammation caused by the immune reaction to the virus. Blood tests may show elevated levels of liver enzymes, a sign of liver damage, which is often the first suggestion that the infection may be present. Patients may become easily fatigued or complain of nonspecific symptoms.

In patients with advanced cirrhosis, the liver begins to fail. This is a life-threatening problem. Confusion and even coma (encephalopathy) may result from the inability of the liver to process certain toxic substances. Increased pressure in the blood vessels of the liver (portal hypertension) may cause fluid to build up in the abdominal cavity (ascites) and result in engorged veins in the swallowing tube (esophageal varices) that tear easily and can bleed suddenly and massively. Portal hypertension also can cause kidney failure or an enlarged spleen resulting in a decrease of blood cells and the development of anemia, increased risk of infection and bleeding.

In advanced cirrhosis, liver failure causes decreased production of clotting factors. Patients with advanced cirrhosis often develop jaundice because the damaged liver is unable to eliminate bilirubin that is formed from the hemoglobin of old red blood cells. Most of the signs and symptoms of HCV relate to the liver. Less commonly, but increasingly, HCV causes conditions outside of the liver, including cryoglobulinemia (production of cryoglobulins), skin conditions (lichen planus and porphyria cutanea tarda), diabetes (primarily type II), platelet destruction (decrease in production of clotting factors), cancer (especially B cell lymphoma, hepatocellular cancer) and Raynaud's disease, among others.

One secondary condition of HCV infection is the production of “cryoglobulins”, which are unusual antibodies produced by the body. These cryoglobulins cause inflammation of the arteries (vasculitis) which may damage the skin, joints, and kidneys. Patients with cryoglobulinemia may have joint pain, arthritis, a raised purple rash on the legs, generalized pain or swelling. In addition, these patients may develop Raynaud's phenomenon, in which the fingers and toes turn color (white, then purple, then red) and become painful at cold temperatures. Two skin conditions, lichen planus and porphyria cutanea tarda, have been associated with chronic infection with HCV. For reasons that are unclear, diabetes is three times more common among patients with chronic HCV infection than in the general population.

Of 100 people infected with HCV, it is estimated that 75 to 85 will become chronically infected, 60 to 70 will develop liver disease, 5 to 20 will develop cirrhosis and 1 to 5 will die from complications of liver disease like cirrhosis or liver cancer.

### Relapse of HCV Infection

Relapsers are patients who initially eliminate the RNA from their blood but then develop detectable RNA again shortly after discontinuing therapy. The RNA becomes detectable again within six months and usually within the first three months of stopping treatment.

When people first acquire HCV, the infection is said to be ‘acute’. There is no standard approach to treatment for acute HCV. Most patients with acute HCV do not have symptoms, so they are not recognized as being infected. However, some have low-grade fever, fatigue or other symptoms that may lead to an early diagnosis. Others who become infected have a known exposure to an infected source, such as a needlestick injury, and are monitored closely. Treatment decisions should be made on a case-by-case basis. However many experts prefer to hold treatment for several months to see whether the patient eliminates the virus without treatment.

HCV is the leading reason for liver transplantation in the U.S., accounting for 40% to 45% of transplants. HCV routinely recurs after transplantation and infects the new liver. Approximately 25% of these patients with recurrent hepatitis will develop cirrhosis within five years of transplantation. Despite these findings of recurrence, the five-year survival rate for patients with HCV is comparable to that of patients who are transplanted for other types of liver disease.

In the U.S., infection with HCV is the most common cause of chronic hepatitis and the most common reason for liver transplantation. HCV is diagnosed by determining levels in the blood of antibodies to the virus and then confirmed with other tests for viral RNA. The amount of viral RNA in the blood (viral load) does not correlate with the severity of the disease but can be used to track the response to treatment. A liver biopsy may be used to assess the amount of liver damage (liver cell injury and scarring), which can be important in planning treatment.

Considerable progress has been made in the treatment of HCV. Combined therapy with pegylated interferon and ribavirin is the present standard treatment regimen, which can be combined with the presently claimed compounds in treating HCV infections. Treatment results in reduced inflammation and scarring of the liver in most sustained responders and also occasionally (and to a much lesser extent) in those who relapse or do not respond. Some HCV infections develop resistance to traditional therapy (interferon, including pegylated interferon and/or ribavirin) and the presently claimed compounds are particularly useful in treating those HCV infections.

The present invention includes compositions comprising the pharmaceutically acceptable salts of compounds of the present invention. The acids and bases which may be used to prepare the pharmaceutically acceptable acid or base addition salts of the aforementioned compounds useful in this invention are those which form base addition salts. The chemical bases that may be used as reagents to prepare pharmaceutically acceptable base salts of the present compounds that are acidic in nature are those that form non-toxic base salts with such compounds. Such non-toxic base salts include, but are not limited to those derived from such pharmacologically acceptable cations such as alkali metal cations (eg., potassium and sodium) and alkaline earth metal cations (e, calcium and magnesium), ammonium or water-soluble amine addition salts such as N-methylglucamine-(meglumine), and the lower alkanolammonium and other base salts of pharmaceutically acceptable organic amines, among others.

Compositions according to the present invention may also include non-toxic acid addition salts, i.e., salts containing pharmacologically acceptable anions, such as the hydrochloride, hydrobromide, hydroiodide, nitrate, sulfate, bisulfate, phosphate, acid phosphate, acetate, lactate, citrate, acid citrate, tartrate, bitartrate, succinate, maleate, fumarate, gluconate, saccharate, benzoate, methanesulfonate, ethanesulfonate, benzenesulfonate, p-toluenesulfonate and pamoate [i.e., 1,1′-methylene-bis-(2-hydroxy-3 naphthoate)]salts, among others.

The compounds of this invention primarily relates to porphyrin compounds as otherwise described herein, but can include other stereoisomers where relevant, including optical isomers of the present compounds, as well as racemic, diastereomeric and other mixtures of such isomers, as well as all solvates and/or polymorphs of the compounds.

The compositions of the present invention may be formulated in a conventional manner in pharmaceutical dosage form using one or more pharmaceutically acceptable carriers and may also be administered in controlled-release formulations. Pharmaceutically acceptable carriers that may be used in these pharmaceutical compositions include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as prolamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. Preferably, the compositions are administered orally, intraperitoneally or intravenously.

Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The pharmaceutical compositions of this invention may be orally administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, the pharmaceutical compositions of this invention may be administered in the form of suppositories for rectal administration. These can be prepared by mixing the agent with a suitable non-irritating excipient which is solid at room temperature but liquid at rectal temperature and therefore will melt in the rectum to release the drug. Such materials include cocoa butter, beeswax and polyethylene glycols.

The pharmaceutical compositions of this invention may also be administered topically, as well. Suitable topical formulations are readily prepared for each of these areas or organs. Topical application for the lower intestinal tract can be effected in a rectal suppository formulation (see above) or in a suitable enema formulation. Topically-acceptable transdermal patches may also be used.

For topical applications, the pharmaceutical compositions may be formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water.

Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

The pharmaceutical compositions may be formulated for ophthalmic use as micronized suspensions in isotonic, pH adjusted sterile saline, or, preferably, as solutions in isotonic, pH adjusted sterile saline, either with our without a preservative such as benzylalkonium chloride.

The pharmaceutical compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

The amount of compound in a pharmaceutical composition of the instant invention that may be combined with the carrier and other materials to produce a single dosage form will vary depending upon the host and disease treated, and the particular mode of administration. Preferably, the compositions should be formulated to contain between about 0.1 milligram to about 750 milligrams, more preferably about 1 milligram to about 600 milligrams, and even more preferably about 10 milligrams to about 500 milligrams of active ingredient.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease or condition being treated.

The porphyrin compound which is formulated and administered to a patient or subject is that amount effective to produce an intended therapeutic result and may vary widely. Preferably, pharmaceutical compositions according to the present invention should be formulated so that a therapeutically effective dosage of between about 0.1 μg/kg and 100 mg/kg, about 0.50 m/kg and 20 mg/kg, about 1 μg/kg and 20 mg/kg about 5 μg/kg to about 15 mg/kg, about 500 μg/kg to about 10 mg/kg patient/day of the compound can be administered to a patient receiving these compositions.

According to one embodiment, it will be appreciated that the amount of a compound of the present invention required for use in treatment will vary not only with the particular compound selected but also with the route of administration, the nature of the condition for which treatment is required and the age and condition of the patient and will be ultimately at the discretion of the attendant physician or veterinarian. The desired dose according to one embodiment is conveniently presented in a single dose or as divided dose administered at appropriate intervals, for example as two, three, four or more doses per day.

In another embodiment, the compound is conveniently administered in unit dosage form; for example containing 1 to 1500 mg, conveniently 20 to 750 mg, most conveniently 25 to 650 mg of active ingredient per unit dosage form.

According to another embodiment of the present invention, the active ingredient is administered to achieve peak plasma concentrations of the active compound of from about 0.5 to about 75 μM, about 1 to 50 μM, about 3 to 30 μM. This may be achieved, for example, by the intravenous injection of a 0.1 to 5% solution of the active ingredient, optionally in saline, or orally administered as a bolus containing about 0.5 to about 500 mg or more of the active ingredient. Desirable blood levels may be maintained by a continuous infusion to provide about 0.01 to about 5.0 mg/kg/hour or by intermittent infusions containing about 0.4 to about 15 mg/kg of the active ingredient.

For use in therapy, a compound according to the present invention may be administered as the raw chemical, although it is preferable according to one embodiment of the invention, to present the active ingredient as a pharmaceutical formulation. The embodiment of the invention thus further provides a pharmaceutical composition comprising a porphyrin compound according to the present invention, or a pharmaceutically acceptable salt thereof together with one or more pharmaceutically acceptable carrier, additive and/or excipient therefor and, optionally, other therapeutic and/or prophylactic ingredients. The carrier, additive and/or excipient must be “acceptable” in the sense of being compatible with the other ingredients of the formulation and not deleterious to the recipient thereof.

The present pharmaceutical formulations include but are not limited to those suitable for oral, rectal, nasal, topical (including buccal and sub-lingual), transdermal, vaginal or parenteral (including intramuscular, subcutaneous and intravenous) administration or in a form suitable for administration by inhalation or insufflation. The formulations may, where appropriate, be conveniently presented in discrete dosage units and may be prepared by any of the methods well known in the art of pharmacy. All methods according to this embodiment include the step of bringing into association the active compound with liquid carriers or finely divided solid carriers or both and then, if necessary, shaping the product into the desired formulation.

According to another embodiment, pharmaceutical formulations suitable for oral administration are conveniently presented as discrete units such as capsules, cachets or tablets each containing a predetermined amount of the active ingredient; as a powder or granules. In another embodiment, the formulation is presented as a solution, a suspension or as an emulsion. Still in another embodiment, the active ingredient is presented as a bolus, electuary or paste. Tablets and capsules for oral administration may contain conventional excipients such as binding agents, fillers, lubricants, disintegrants, or wetting agents. The tablets may be coated according to methods well known in the art. Oral liquid preparations may be in the form of, for example, aqueous or oily suspensions, solutions, emulsions, syrups or elixirs, or may be presented as a dry product for constitution with water or other suitable vehicle before use. Such liquid preparations may contain conventional additives such as suspending agents, emulsifying agents, non-aqueous vehicles (which may include edible oils), or preservatives.

The compounds of the present invention according to an embodiment are formulated for parenteral administration (e.g. by injection, for example bolus injection or continuous infusion) and may be presented in unit dose form in ampoules, pre-filled syringes, small volume infusion or in multi-dose containers with an added preservative. The compositions may take such forms as suspensions, solutions, or emulsions in oily or aqueous vehicles, and may contain formulatory agents such as suspending, stabilizing an/or dispersing agents. Alternatively, the active ingredient may be in powder form, obtained by aseptic isolation of sterile solid or by lyophilisation from solution, for constitution with a suitable vehicle, e.g. sterile, pyrogen-free water, before use.

For topical administration to the epidermis, the compounds, according to one embodiment of the present invention, are formulated as ointments, creams or lotions, or as a transdermal patch. Such transdermal patches may contain penetration enhancers such as linalool, carvacrol, thymol, citral, menthol and t-anethole. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or colouring agents.

Formulations suitable for topical administration in the mouth include lozenges comprising active ingredient in a flavoured base, usually sucrose and acacia or tragacanth; pastilles comprising the active ingredient in an inert base such as gelatin and glycerin or sucrose and acacia; and mouthwashes comprising the active ingredient in a suitable liquid carrier.

Pharmaceutical formulations suitable for rectal administration wherein the carrier is a solid. In another embodiment, they are presented as unit dose suppositories. Suitable carriers include cocoa butter and other materials commonly used in the art, and the suppositories may be conveniently formed by admixture of the active compound with the softened or melted carrier(s) followed by chilling and shaping in moulds.

According to one embodiment, the formulations suitable for vaginal administration are presented as pessaries, tampons, creams, gels, pastes, foams or sprays containing in addition to the active ingredient such carriers as are known in the art to be appropriate.

For intra-nasal administration the compounds, in one embodiment of the invention, are used as a liquid spray or dispersible powder or in the form of drops. Drops may be formulated with an aqueous or non-aqueous base also comprising one more dispersing agents, solubilising agents or suspending agents. Liquid sprays are conveniently delivered from pressurized packs.

For administration by inhalation the compounds, according to one embodiment of the invention are conveniently delivered from an insufflator, nebulizer or a pressurized pack or other convenient means of delivering an aerosol spray. In another embodiment, pressurized packs comprise a suitable propellant such as dichlorodifluoromethane, trichlorofluoromethane, dichlorotetrafluoroethane, carbon dioxide or other suitable gas. In another embodiment, the dosage unit in the pressurized aerosol is determined by providing a valve to deliver a metered amount.

Alternatively, in another embodiment according to the present invention, for administration by inhalation or insufflation, the compounds according to the present invention are in the form of a dry powder composition, for example a powder mix of the compound and a suitable powder base such as lactose or starch. In another embodiment, the powder composition is presented in unit dosage form in, for example, capsules or cartridges or e.g. gelatin or blister packs from which the powder may be administered with the aid of an inhalator or insufflator.

In an additional embodiment according to the present invention, any one or more of the above described formulations are adapted to provide sustained and/or controlled release of the active ingredient.

The compounds of the invention may also be formulated and used in combination with effective amounts of other antiviral agents, including for example, interfereon (IFN, including pegylated IFN), ribavirin, BILN 2061, G418, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9 Agonist, PHX1766, SP-30 and mixtures thereof.

Methods of treating, inhibiting and/or reducing the likelihood of virus infections represent additional aspects of the present invention. In a first method embodiment, the viral infection is chosen from Flaviviridea viral infections. In another method embodiment, the Flaviviridea viral infection is chosen from Hepatitis C virus (HCV), bovine viral diarrhea virus (BVDV), hog cholera (swine fever, yellow fever, dengue fever and West Nile virus (WNV).

In a preferred embodiment, the Flaviviridea viral infection is Hepatitis C.

### EXAMPLES

The following examples are provided to further describe the present invention. These examples are for illustration only and should not be taken to limit the invention in any way.

### Materials and Methods

Materials. Meso-Tetra(4-carboxyphenyl)porphine (compound 1) was purchased from Frontier Scientific, Inc. The synthesis of compounds 4, 6 and 1 were described in a previous publication (1) and the chemical synthesis procedures of the other analogues can be found in the supplementary materials. 5,10,I5,20-Tetrakis (4(trimethylammonio)-phenyl)-21H,23H-porphine (TTMAPP) and 4,4′,″,4″ (Porphine-5,10,15,20-tetrayl)tetrakis(benzenesulfonic acid) tetrasodium salt hydrate (TPPS4) were purchased from Sigma-Aldrich. NS3/44 protease inhibitor BILN 2067, developed by Boehringer Ingelheim (19), was a kind gift from Tsu-an Hsu from the National Health Research Institutes, Taiwan.

Cells. HCV genotype 1b (Con 1 isolate) subgenomic replicon cell line with luciferase reporter (Huh-luc/neo-ET) was kindly provided by Ralf Bartenschlager from the University of Heidelberg (37). Huh-luc/neo-ET cells were cultured in Dulbecco's modified Eagle medium (DMEM , supplemented with 10% fetal bovine serum (FBS), 1 mM nonessential amino acids, and 250 μg/ml of G418 (Invitrogen). Genotype 2a (JFH-I isolate) subgenomic replicon cells YSGR-JFH were cultured in DMEM containing 10 To fetal calf serum, 1 mM nonessential amino acids and 400 μg/ml G418 (31). An additional genotype 1b (Conl) replicon cell line (4291BBix) was cultured in similar media but with 3 μg/ml blasticidin (22).

Determination of antiviral activities. Ff-luciferase reporter activity was used to monitor the replication of HCV replicons in Huhluc/neo-ET cells free from G418. Replicon cells were seeded at the density of 5×10³ cells per well in 96-well plates. The following day, replicon cells were incubated in duplicates with DMSO or serially diluted tetraphenylporphyrin (TPP) analogues at 37″C. 72 hrs after co-incubation, cells were lysed with ice-cold passive lysis reagent after PBS wash, and the luciferase activity was measured with the luciferase assay kit (Promega) and Tecan FARCyte luminometer (GE Healthcare) following the manufacturers' descriptions. The relative light units (RLU) were adjusted as percentage readings of the compound-free controls and the 50% effective concentration (EC₅₀) was determined from dose-effect curve by nonlinear regression analysis using Origin 6.1 (OriginLab Software). TPP analogues were also screened in vitro against HIV-I IIIB and HBV as previously described (26,41). Briefly 1×10³ MT-2 cells per well were exposed in triplets to 0.1TC1D₅₀/cell (50% of the tissue culture infectious dose) of HIV-1 IIIB and cultured in the presence of compounds. The EC₅₀ values were estimated by MTT-based colorimetric quantitation of viral CPE after 5 days. Anti-HBV activities were evaluated in 2.2.15 cells by means of Southern DNA hybridization.

Determination of cytotoxicity and mitochondrial DNA toxicity. Exponentially growing Huh-7 cells were seeded at a density of 1×10⁴ cells per well in a 24-well plate and incubated with TPP analogues for 3 days. Cells were fixed and stained with 0.5% methylene blue in 50% ethanol followed by extensive washing. After the plates were air dried, cells were solubilized in 1% sarkosyl and cell growth was determined from the extent of absorption by spectrophotometric measurements at 595 nm (Biotek Instruments). For compounds whose dark color interfered with 595-nm reading, cytotoxicity was measured by the CellTiter-Glo luminesecent cell viability assay following manufacturer's description (Promega). Cytotoxicity in MT-2 cells was determined from colorimetric quantitation of uninfected MT-2 cells after 5 days of coincubation.

Quantitation of mitochondrial DNA content was performed as previously published (18). Briefly, CEM cell lysates were spotted onto Hybond paper using the Miliford II slot blot apparatus (Schleicher & Schuell). MTDNA was detected with mtDNA-specific probe and then reprobed with Alu probe for internal control. The autoradiographic bands were quantified on scanning densitometer (Molecular Dynamics).

The impact of serum concentration in culture media on the antiviral activities of compound 6 and its analogues. Similar to the HCV replicon assay, Huhluc/neo-ET cells were incubated with serially diluted compounds in the presence of 5%, 10%, 20%, or 40% (v/v) FBS. Cells were harvested after 72 hrs for luciferase activity assays.

Quantification of HCV RNA and NS5A protein. Huhluc/neo-ET cells were seeded at the density of 1×10⁵ cells per well in 6-well plate and treated with either DMSO control or up to 250 nM of compound 6. Cells were harvested after 24, 48, or 72 hrs of incubation and subjected to luciferase activity assay, RNA quantification and immunoblotting. Results were averaged from three independent repeats. Luciferase activity assay was done in triplets per experiment as aforementioned and RLU reading was normalized against the total protein level per sample determined from Bradford assay. Total RNA was isolated using RNeasy Mini Kit (QIAGEN) and the RNA concentrations were measured by spectrophotometry (GE Healthcare) followed by dilution into 50 ng/μL. Replicon RNA was quantitated in triplicates by amplifying the HCV 5′UTR using one-step real-time reverse transcriptase polymerase chain reactions (qRT-PCR). Each 20 μL replicate contained 100 ng of total RNA, 100 nM probe (6FAM-5′-TATGAGTGTCGTACAGCCTCCAGG-3′-MGBNFQ, Applied Biosystems) and 200 nM forward and reverse primers (5′-CTTCACGCAGAAAGCGTCTA-3′, and 5′-CAAGCACCCTATCAGGCAGT-3′ respectively, Yale University W.M. Keck Facility) (21), together with iScript reverse transcriptase and reaction mix for one-step RT-PCR (Bio-Rad Laboratories). Reactions were run in the iCycler iQ RealTime thermocycler detection system (Bio-Rad Laboratories) as follows: 10 min at 50° C., 5 min at 95° C., followed by 42 cycles at 95° C. for 15 sec and 60° C. for 30 min. Results were normalized against the β-actin mRNA levels in each sample (20).

For immunoblot analyses, cells were lysed in 100 μL of lysis/loading buffer (30 mM Tris 6.8, 12.5% glycerol, 1% SDS, 5% β-mercaptol ethanol, and 0.01% bromophenol blue). Samples were electrophoresed by 8% SDS-PAGE and transferred onto a nitrocellulose membrane for 30 min at 15V using Trans-Blot semi-dry transfer apparatus (Bio-Rad Laboratories). The membrane was blocked with 5% non-fat dry milk in PBS for t hr and probed by mouse monoclonal antibody (7D4) specific for Hepatitis C Virus NS5A (Santa Cruz Biotechnology, Santa Cruz, Calif.) or monoclonal antibody specific for human α-tubulin (Sigma-Aldrich) at 4° C. overnight followed by washing in PBS with 0.2% Tween 20. After incubation with goat anti-mouse Ab (Sigma-Aldrich) for 1 hr at room temperature, the membrane was washed extensively and detected by chemiluminescent procedures according to manufacturer's instructions (Perkin Elmer).

Reversibility of the action of compound 6 against genotype 1b HCV replicon. Huh-luc/neo-ET cells were seeded at a density of 2×10⁵ cells per well in a 6-well plate, and were incubated for 12 days with DMSO control or up to 1 μM of compound 6 in the absence of G418. Cells were split every three days when media and compounds were replenished, and samples were collected for RNA quantification. compound 6 was removed on Day 12 when cells were split and cultured in the presence of 250 μg/ml of G418. Replicon cells were continuously monitored for another 12 days, during which cells were split and sampled whenever reaching confluence. Cell viability was measured by CellTiter-Glo luminescent cell viability assay following manufacturer's procedures (Promega), and the HCV RNA was quantitated by qRT-PCR and normalized as described above.

Activity of compound 6 against genotype 2a (JFH-I) replicons. In parallel to Huh-luc/neo-ET cells, 1×10⁵ YSGR-JFH cells per well were incubated with DMSO control, compound 6, or recombinant human IFNα-2a (Pestka Biomedical Laboratories) in 6-well plates. Cells were harvested after 72 hrs of co-incubation and HCV RNAs were quantitated by qRT-PCR and normalized as described above.

Combination studies. Huh-luc/neo-ET cells were seeded at the density of 5×10³ cells per well in 96-well plates. In the following day a mixture of two components (compound 6 with IFNα-2a, or compound 6 with BILN 2061) were applied in serial dilution and hence kept at constant ratio. A total of eight different mixtures were assayed in duplicates such that the potency ratio of the two compounds ranged from emphasizing Drug 1/de-emphasizing Drug 2 to de-emphasizing Drug 1/emphasizing Drug 2.72 hrs after co-incubation, cells were harvest for luciferase activity assay and the median-effect equation was used for dose-effect analysis. The doses of Drug 1 and Drug 2 required to inhibit HCV replication by x % when used alone were denoted as (Dₓ)₁ and (Dₓ)₂, whereas the apparent isoeffective doses needed to achieve xVo inhibition when used in combination were denoted as (D)₁ and (D)₂. The ratios (D)₁/(Dₓ)₁ were plotted against (D)₂/(Dₓ)₂ in antiviral isobolograms, in which the hypotenuse represents the line of additivity. If the experimental isobole bows below the hypotenuse, the combination is considered to be synergistic; if the isobole bows above the hypotenuse, antagonism is suggested (5, 13, 33). P value was determined by two-way ANOVA test using GraphPad Prism 4.0.

### Results

### Structure-Activity Relationships of the Tetraphenylporphyrin Analogues.

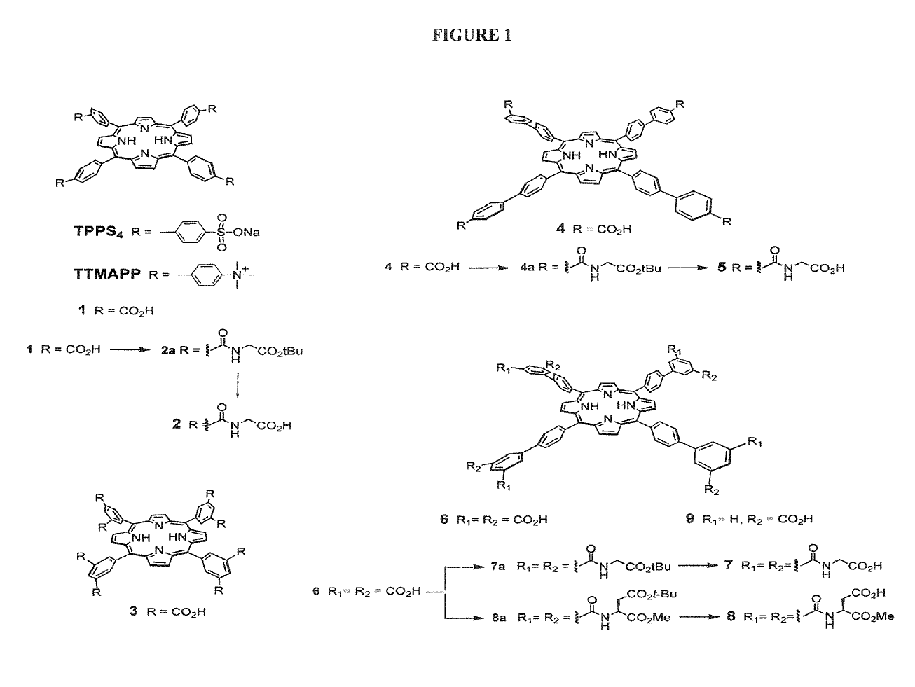

From a small library of porphyrin analogues that the inventors initially explored for antiviral application, compound 2 emerged as a micromolar inhibitor of HCV replicons in vitro and provided the first insight into the development of meso-tetrakis-phenylporphyrin (TPP) derivatives as anti-HCV agents (Table 1, see below). Over 7-fold improvement in the anti-HCV EC₅₀, together with a decrease of cytotoxicity and less effect on mitochondrial DNA synthesis, was observed in its synthetic precursor compound 1 in which the aspartic acid side chains were replaced with more rigid and planar carboxylic acids. Substitution of the carboxylic acids in compound L with sulfonic acids as in TPPS+ led to complete loss of anti-HCV activity in vitro (EC₅₀ 51.24×8.577 μM). Reversing the charge of functional groups in the example of TTMAPP also compromised the activity against HCV (EC₅₀ 3.58+0.208 μM). The structures of the TPP analogues are shown in FIG. 1.

With the goal of improving hydrophobic surface recognition, we extended each peptidic appendage by one phenyl ring giving rise to the family of meso-tetrakisbiphenylporphyrins (TBPs) that included compounds 4 and 5. The distance from opposite para positions of the phenyl groups was thus extended from approximately 15.5 Å in TPPS to 24.0 Å in TBPs, thereby increasing the total recognition area by 100 Å² (34). Expansion of surface area from compound 2 to compound 5 was accompanied by a 12-fold improvement in anti-HCV EC₅₀. However, this was not the case in the comparison of compound 1 to its larger homolog 4 which showed reduced the antiviral activity.

The inventors then increased the total negative charges of the peripheral groups from four to eight in order to enhance electrostatic interactions with potentially complementary regions on the target(s). While compound 3 was relatively ineffective against HCV replicons, compound 6 proved to be the most potent nanomolar inhibitor in our study, with an EC₅₀ of 0.024+0.0051 μM that represented a 75-fold improvement over the lead compound 2. Given the extremely low cytotoxicity in naïve Huh-7 cells, compound 6 offers a favourable selective index in culture (CC₅₀/EC₅₀) of over 2000. Additionally, compound 6 did not alter the amount of mitochondrial DNAs. Removal of one carboxylic acid from the meta position on each phenyl ring gave rise to compound 9 with decreased antiviral activity, suggesting that the projection of all eight negative charges are indispensable for potent inhibition of HCV replicons. As the functional groups became bulkier and more flexible in the cases of compounds 7 and 8, the antiviral activity was substantially decreased. To study if metallation of the porphyrin rings can influence the antiviral function, we synthesized zinc-, copper- and iron-conjugates of compound 6 and compared their EC5ss. Results suggested that metallation of porphyrin core did not significantly alter the anti-HCV activity (See Table 3 below).

In addition to anti-HCV SAR, we also examined the nine TPP analogues against HBV and HfV-1 IIIB to establish antiviral specificity (Table 1). None of the compounds were able to inhibit IIBV; whereas analogues bearing tetrabiphenylporphyrin motifs (compounds 5-8) exhibited micromolar activity against HIV-I IIIB.

### Compound 6 Suppressed Viral Macromolecules in Genotype 1b Replicon Cells in a Time-Dependent and Dose-Dependent Manner.

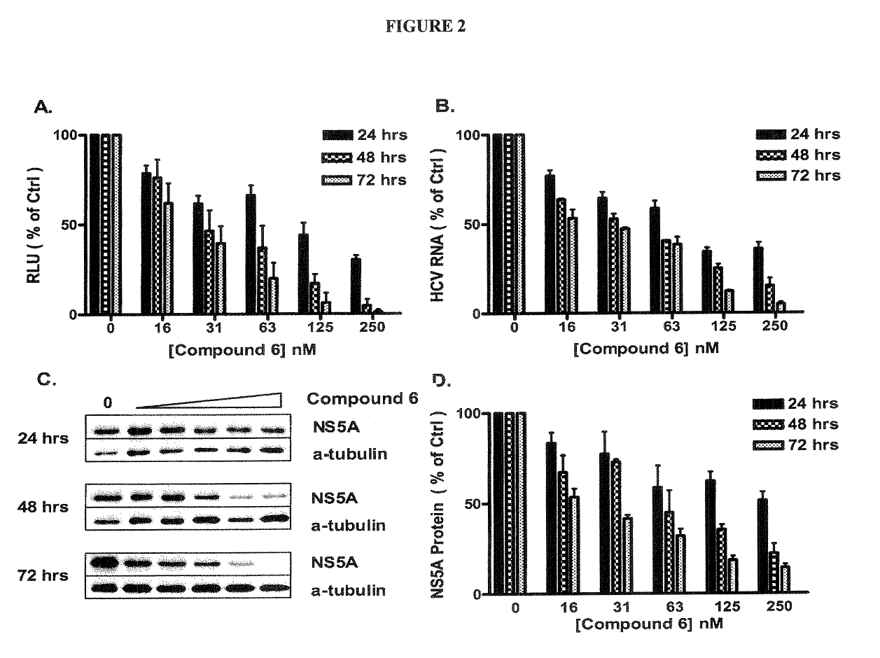

The anti-HCV activities of compound 6 in Huh-luc/neo-ET cells were characterized by studying different viral parameters: the luciferase reporter activity, HCV RNA level, and the protein level of NS5A (FIG. 2). The replicon luciferase activity was markedly inhibited by compound 6 in a dose-dependent manner and the EC₅₀s decreased with incubation time indicating improved efficacy. The 24-hr and 48-hr EC₅₀s of compound 6 were 57.8 nM and 19.2 nM respectively; the 72-hr EC₅₀ was 17.6 nM, an over 3-fold improvement compared with that of 24-hrs incubation (FIG. 2A). Since luciferase activity indirectly reflects the overall level of viral replication, the inventors expected the HCV RNA to be suppressed in a similar fashion post exposure to the inhibitor. Quantitative amplification of the HCV 5′-UTR demonstrated that compound 6 indeed led to a reduction of the HCV RNA level in replicon cells in a time- and dose dependent manner (FIG. 2B). The relative HCV RNA level was expressed as percentage of the mock-treated control. The 24-lu and 48-hr EC₅₀s of compound 6 were estimated to be 67.8 nM and 36.2 nM respectively; the 72-fu EC₅₀ was 29.2 nN·4, an over 2-fold improvement compared with that of 24-hr incubation. When viral protein amount was quantitated by western blot analyses, a similar reduction of the NS5A protein level in drug-treated replicon cells was observed (FIGS. 2C and D).

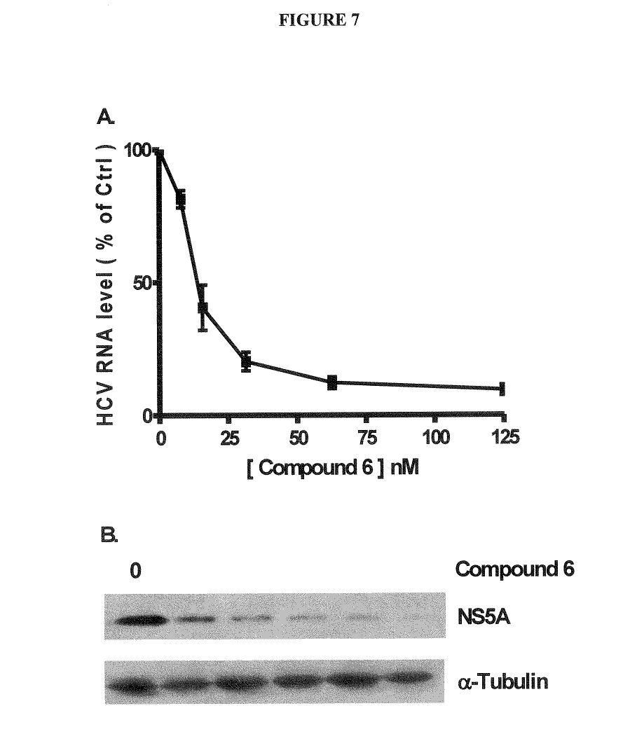

The antiviral activities were confirmed in 429/BBix, a Huh-7.5 cell line carrying genotype 1b replicon that confers blasticidin resistance and does not carry luciferase reporter gene (FIG. 7). This also ruled out the possibility that compound 6 exerted its action by interacting with non-viral components of the replicon, i.e., Ff-luc, neo′ genes and their gene products.

The Effect of Serum Concentration in Culture Media on the Anti-HCV Activity of Compound 6 & its Analogues.

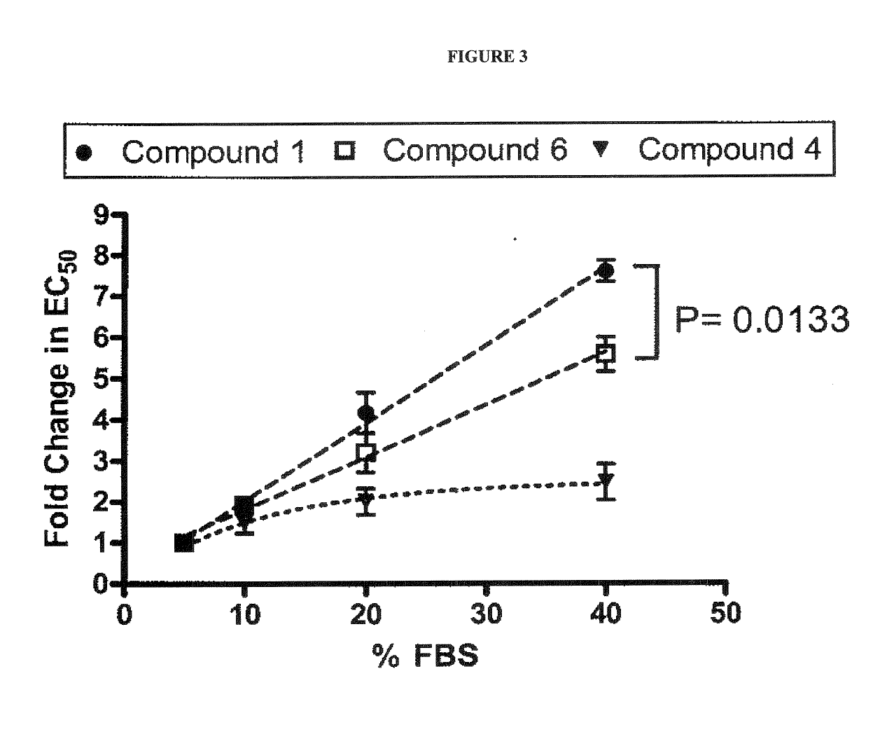

Sequestration of compounds by serum proteins could decrease the availability of free agent but might also improve the uptake of hydrophobic derivatives. Hence the inventors studied the effects of serum protein binding on the antiviral activity of compounds 1, 4 & 6 using different amounts of FBS in the media (FIG. 3). Huh-luc/neo-ET cells were incubated with compound 1, 4 or 6 for 72 hrs and the anti-HCV activity was evaluated by measuring the reduction of reporter luciferase activities. Up to 40% (v/v) FBS in media did not alter the luciferase activity of Huhluc/neo-ET cells. In all cases the EC5es increased with percentage FBS, which could reflect a decrease of available compounds due to sequestration by serum proteins. Extrapolation of the plot of EC₅₀ vs serum amount provided an estimate of the theoretical EC₅₀ at 0% FBS as shown in Table 2 (below). Comparison of the relative fold changes in EC₅₀ with increasing percentage of FBS showed that compound 1 was more affected by serum binding than compound 6. Compound 4 appeared to be least affected by serum concentration and the fold increase was non-linear unlike the other two analogues.

The Activity of Compound 6 Against Genotype 1b (ConL) Replicon was Reversible but Longer Treatment with Higher Dosages Could Prevent Viral Rebound.

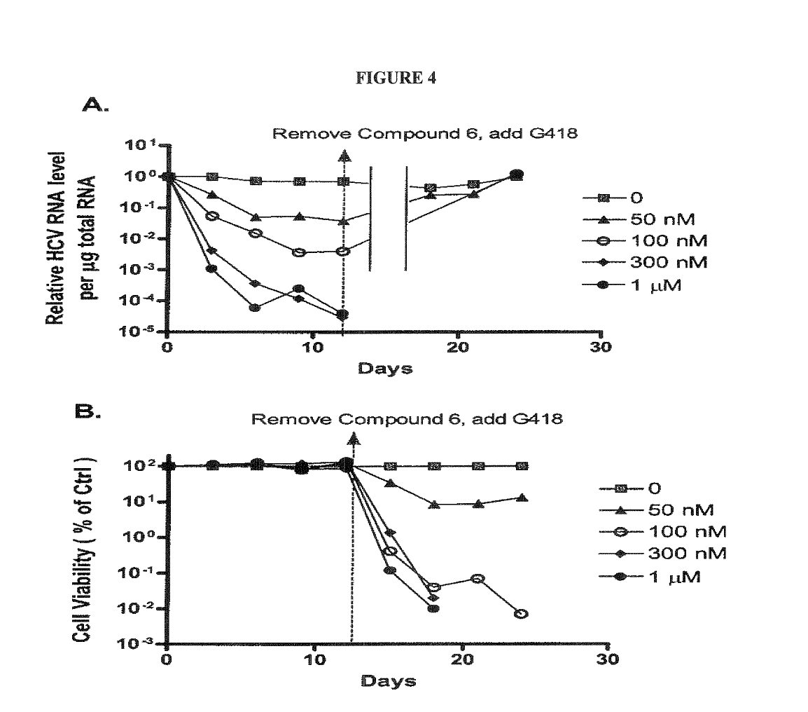

The goal of anti-HCV treatment is to completely eliminate the virus from infected cells. In order to assess the reversibility of the antiviral action and the possibility of replicon clearance, we incubated Huhluc/neo-ET cells with increasing concentrations of compound 6 for 72 days free from G418 (FIG. 4). On Day 12 compound 6 was removed and 250 μgml G418 was reintroduced while the replicon RNA level and cell growth were monitored for another 12 days. Due to the inhibition of viral replication by compound 6, cells that have lower levels of replicons should become more sensitive to G418. Therefore the percentage of cells killed reflected the percentage of cells “cured” by compound 6.

Up to 1 μM compound 6 did not cause significant toxicity in cells during the 12-day treatment. A steady decrease in viability was initially observed when replicon cells were co-cultured with G418, followed by gradual rebound (FIG. 4B). Cells that were treated with higher concentrations of compound 6 experienced significant delay in rebound. 8.5% of the cells treated with 50 nM of compound 6 survived 6 days after the removal of inhibitor and replicon-positive cells slowly rebounded to 13.1% o after a lapse of another 6 days. Only 0.007% of the cells treated with 100 nM of compound 6 survived under the selective pressure of G418 by the end of the experiment; no rebound was observed and the replicon RNA level once fell beneath detection limit. Cells that were exposed to 300 nM and 1 μM of compound 6 were no longer viable 9 days after coincubation with G418, which indicated complete “cure”. Concentrations of compound 6 of 300 nM and above induced approximately 4.5-1o916 reduction in the HCV RNA levels after 12 days of exposure. HCV RNA level in cells treated with lower concentrations of compound 6 rebounded faster than in cells treated with higher dosages (FIG. 4A).

In a separate experiment the inventors treated the replicon cells with compound 6 for 9 days followed by a 15-day rebound period in the presence of G418. Only 0.4% o of the cells treated with 300 nM of compound 6 survived with a lack of rebound and 1 μM of compound 6 achieved complete “cure” (data not shown).

Genotype 2a (JFH-1) Replicon Cells were More Resistant to Both Compound 6 and IFNα-2a.

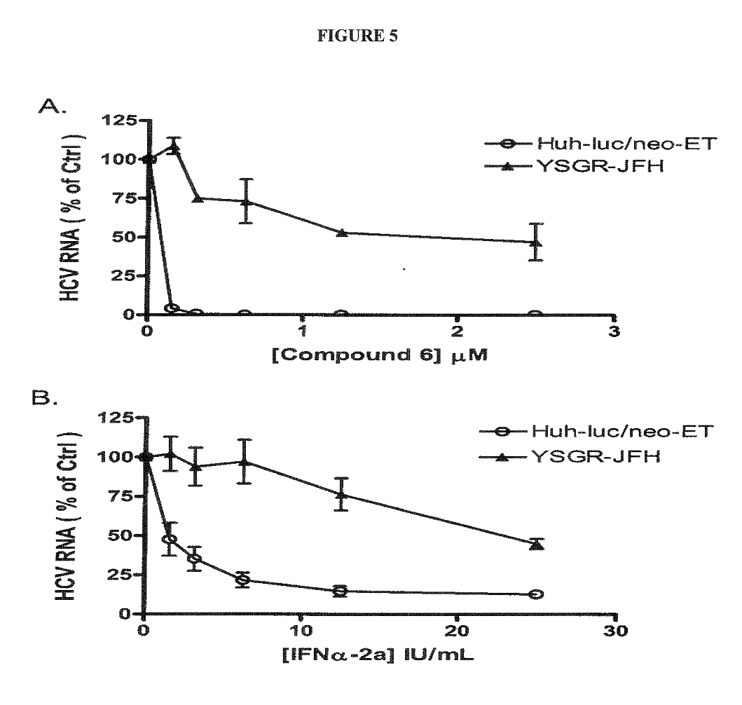

In contrast to Huh-luc/neo-ET replicon cells, YSGR-JFH—a genotype 2a JFH-1 isolate replicon cell line—was more resistant to treatment with compound 6 as well as IFNα-2a. Replicon cells of the two genotypes were incubated with various concentrations of compound 6 or IFNα-2a for 72 hrs and the HCV RNA level was quantitated by qRTPCR. The antiviral EC₅₀ of compound 6 against YSGR-JFH replicon cells was 1.38±0.148 μM, which was over 57-fold higher than the EC₅₀ against Huh-luc/neo-ET replicons (FIG. 5A). The antiviral EC₅₀s of IFNα-2a were 2.39+1.765 IU/mL and 25.99+4.119 IU/mL in Huh-luc/neo-ET and YSGR-JFH replicon cells respectively, representing an approximately 11-fold difference (FIG. 5B). YSGR-JFH replicon cells were also less responsive to compound 1 as expected (EC₅₀ 1.9 μM).

Compound 6 exhibited additive to synergistic effect when combined in vitro with BILN 2061 or IFNα-2a.

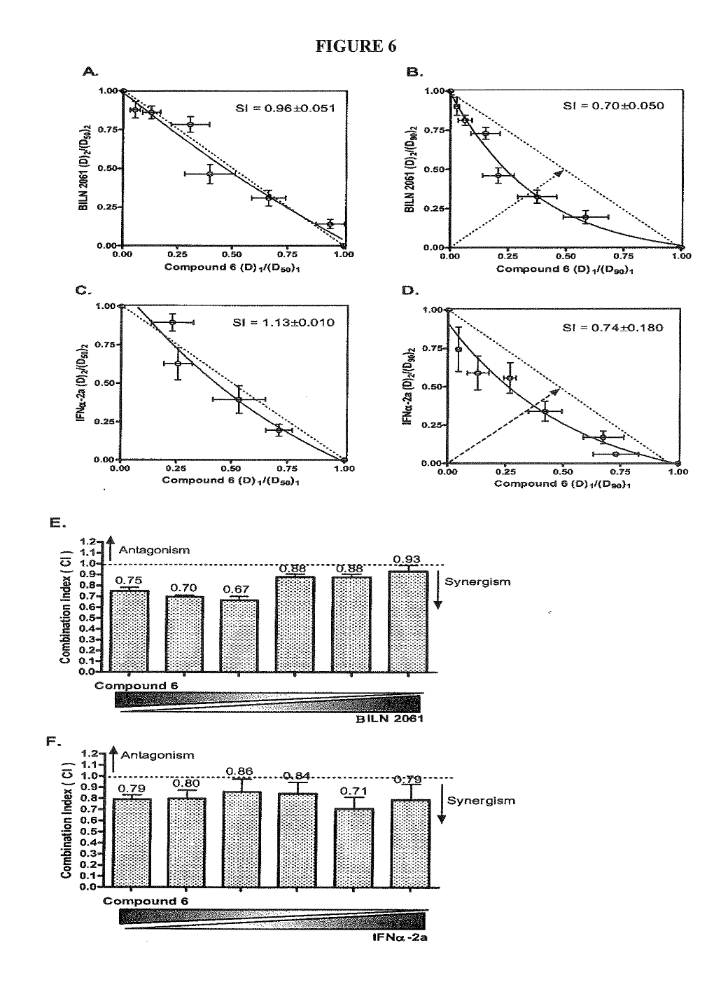

The development of more effective and nontoxic combinations of therapeutic agents has become an important goal in the management of HCV infection. The inventors assessed the combination of compound 6 and established anti-HCV agents with respect to their antiviral activities when used alone. In a classical isobologram, the synergy index (SI) represents the fractional distance from the origin to where the isobole and hypotenuse intersect. Hence SI>1 indicates antagonism and SI<1 synergism. The smaller the SI value the higher the degree of synergism. The intersection also represents the most optimum potency ratio (theoretically (D)₁(Dₓ)₁=(D)₂/(Dₓ)₂) to achieve the highest degree of synergy at a given effect level when the isobole is of symmetrical distribution. As shown in FIGS. 6A and 6C, the combination of compound 6 with BILN 2061 or IFNα-2a was near additive at the 50% effect level (EC₅₀), with SI values around 1.00. Synergistic effect became more apparent at the 90% inhibition level when SI value was around 0.70 and the two isobols differed significantly as indicated by P value <0.0001 (FIG. 68, D). The combination of compound 6 with BILN 2061 (optimum potency ratio=0.34:0.36) was slightly more synergistic than combination with IFNα-2a (optimum potency ratio=0.38:0.37). Calculation of the combination index (CI=(D)₁/(D)₁+(D)₂/(Dₓ)₂) (5) provided a similar conclusion on drug combination: CI₉₀ values of compound 6 in combination with BILN 2061(FIG. 6E) or IFNα-2a (FIG. 6F) confirmed additive to synergistic interactions between the compounds.

### Discussion

In recent decades, efforts in medicinal chemistry have been concentrated in the development of small molecule inhibitors that are selective and high-affinity binders of active sites in the protein cavities with the goal of disrupting protein-protein or protein-ligand interactions. In contrast, the protein exterior surfaces frequently employed in specific recognition during intermolecular interactions have been less explored. Specific targeting of such large interfacial areas with their complex topological distribution of hydrophobic, polar and charged residues can potentially be achieved by molecules that mimic protein surface structures. Porphyrins, peptidocalixarenes and a-helical mimetics are examples of macromolecules that have been designed to bind to protein surface and modulate protein-protein interactions (for a review, see ref (9)) Porphyrins are attractive macrocyclic scaffolds due to their intrinsic compatibility with the biological milieu and their physicochemical properties along with synthesis procedures are also well documented. The photoinactivation of viruses by tetrapyroles has been widely studied. Porphyrins and metalloporphyrins have also demonstrated light independent activity in the micromolar range against HIV and vaccinia virus (7, 38). In particular anionic tetrapyrroles including sulfonated porphyrins such as metallo-TPPS₄ were shown to inhibit HIV-I infection by blocking cell fusion induced by the envelope protein and also possibly by disruption of gp120-CD4 binding (38). Interestingly, an uncharged molecule TPP[2,6-(OH)₂] was equally active against vaccinia virus suggesting that the interaction between charged groups may not be the sole basis for its antiviral activity (3). Exploration of the four-fold symmetry of porphyrin derivatives is best illustrated in the rational design of tetraphenylporphyrins to reversibly block the conductance of voltage gated potassium channels, which are homotetrameric molecules essential for numerous cellular functions. As synthetic mimics of peptide toxins, these cationic porphyrins appear to bind the channel pore and also mediate polyvalent interactions with the conserved acidic residues on the channel subunits (12).

For the development of HCV enzyme-specific therapies, viral protease NS3/44 and RdRP NS5B are the most intensely exploited targets. Successful examples of small molecule inhibitors include protease inhibitor telaprevir (VX-950) and boceprevir (SCH503034), nucleoside polymerase inhibitor R7128 and non-nucleoside NS5B inhibitor VCH-222. The macrocyclic inhibitor of NS3-BILN 2061, despite being suspended in clinical development, is a proof-of-principle peptidomimetic compound that was designed to mimic the conformation of substrate-based hexapeptides bound to NS3 and is active both in vitro and in vivo (19, 36). In the present study we report the development of tetracarboxyphenylporphyrins for feasible interaction with biomolecules involved in HCV replication. This class of tetraphenylporphyrins (TPPs) offers a rigid scaffold capable of forming hydrophobic interactions with protein exteriors or solvent-exposed shallow clefts. The binding of the synthetic ligands could be further strengthened through electrostatic interactions with the cationic groups on the targets (FIG. 1). The inherent four-fold symmetry of TPPs can potentially lead to simultaneous binding to several components/subunits of a heteromeric or homomeric complex. The structural features of TPPs could be of particular interest in antiviral drug discovery, because the virus would require multisite mutations (possibly spanning more than one target protein) to become highly resistant, an event with significantly lower probability than single-site mutation that is often sufficient for conferring resistance to small molecule inhibitors.

Based on a lead, compound 2, that exhibited micromolar activity against HCV replicons, the inventors explored TPP analogues with different structural features in search of a selective inhibitor active in the low nanomolar range. The following key factors were taken into consideration during our structural optimization: (1) surface area, (2) charge, size and flexibility of the peptidic appendages, (3) the projection of functional groups relative to the hydrophobic core, and finally (4) solubility and serum sequestration. An interesting feature of tetraphenylporphyrin (TPP) and tetrabiphenylporphyrin (TBP) derivatives is that the first phenyl ring is perpendicular to the porphyrins core whereas the second phenyl ring lies perpendicular to the first ring and in the same plane as the porphyrin core. Consequently compounds 1 and 4, for example, represent a completely different projection of anionic appendages. As shown in Table 1, structure activity relationship SAR analysis of anionic tetraphenyl porphyrin analogues revealed that the most optimum structure against HCV in vitro is that of an octaanionic tetrabiphenylporphyrin compound 6 (EC₅₀ 0.024±0.0051 μM), which represented a 75-fold improvement in EC₅₀ over the lead compound and is comparable to other anti-HCV agents developed to date. Moreover the carboxylic acids could not be replaced with sulfonate, trimethylammonium, the more flexible aspartic acid, or bulkier moieties, nor could the number of negative charges be decreased—all of which led to a reduction in activity. Metallated derivatives of compound 6 demonstrated anti-HCV activity similar to the parent compound, suggesting that contact with the porphyrin core does not contribute towards anti-HCV activity, or compound 6 itself becomes metallated upon entering the cells. Expansion of the hydrophobic surface area improved antiviral efficacy except in the case of compound 1 to 4, which may be due in part to their different projections of anionic groups and their differences in serum binding. Sequestration by serum has the potential to decrease the availability of free drug, but may also improve its solubility and promote uptake into the hepatocytes. Compound 1 appeared to have the highest degree of binding to serum proteins and its anti-HCV EC₅₀ increased linearly with percentage serum in the media (FIG. 3, Table 2). In contrast, compound 4 has the lowest degree of serum association, which could hinder its uptake. Sharing the same hydrophobic core, compound 6 however benefits from a greater number of electrostatic interactions that could help towards uptake into cells. The activity of compound 6 against HCV replicons was confirmed by the suppression of viral RNA and protein levels of two independent genotype 1b (Conl) replicons established in Huh 7 and Huh 7.5 cells respectively (FIG. 2).

Compounds 1, 4, 5, 6 and 9 are selective inhibitors of HCV in vitro and are relatively inactive against DNA virus HBV and RNA virus HIV-I IIIB. Although compounds 5, 6 and their bulkier derivatives showed micromolar inhibition of HIV-I IIIB comparable to tetraporphines that are under development as microbiocides, there was no correlation between the trend of anti-HCV and anti-HIV efficacy therefore it is unlikely that the two types of antiviral activities share the same mechanism of action. Hamilton had previously shown that tetracarboxyphenylporphyrin derivatives bind cytochrome c. Compounds 1, 4, 6 and 8 were found to bind cyt c with K_{d} values of 0.95±0.25, 17±0.84, 1.5±0.17 and 1.7±0.097 μM, respectively (1, 16), but this property did not correlate with the SAR in the present study. We treated Huh-luc/neo-ET cells with up to 1 M of compound 6 for 9 days, during which the media were replenished every 3 days and the cells were passaged once. Live cells were stained with the ratiometric indicator JC-1 (Invitrogen) in order to measure the mitochondrial potential using confocal microscopy. Compared with mock-treated control, compound 6 did not affect the mitochondrial potential unlike the classical uncoupler valinomycin (Calbiochem). In light of the extremely low toxicity on cells and particularly on the amount of mitochondrial DNA, it is unlikely that the potent anti-HCV activity of compound 6 is mediated through cyt c binding.

Compared with subgenomic genotype 1b (Conl) replicons, genotype 2a (JFH-I) replicon appeared to be more resistant to IFNα-2a with 11-fold difference in the anti-HCV EC5e, in accordance with literature (27). Conl and JFH-I isolate differ significantly in their replicase coding region. Surprisingly genotype 2a (JFH-1) replicon was also more resistant to compound 6 and the anti-HCV EC₅₀ fell into micromolar range, being 57 times less effective than the activity against genotype 1b (Conl) replicons. The HCV RNA levels were similar between the two cell lines indicating that differences in replication capacity could not be the major contributing factor. Besides the significant impact of genetic variability on the drug sensitivity, the observed differential response to IFNα-2a and compound 6 between the two subgenomic replicons could be correlated. HCV is known to suppress host immune responses and reduction of viral load restores the production of IFNc/B and related antiviral signalling pathways (10, 30). Therefore the antiviral activity exerted by compound 6 could be augmented through the action of revived host defences and the IFN amplification loops. Such amplification could be more significant in genotype 1b (Conl) replicon-containing cells due to their intrinsic IFN sensitivity and this potential “dual inhibition” could be masked in cells harbouring genotype 2a (JFH-1) replicons.

Unlike the treatment of HIV, HCV therapy can lead to complete eradication of virus in a significant proportion of patients. The present inventors have demonstrated that the antiviral activity of compound 6 was irreversible if the treatment period is sufficiently long and the dosages adequate (FIG. 4). Moreover the longer the treatment, the further was the delay in viral rebound. The limitation of the replicon model, however, could be the relationship between the viral load per cell and the sensitivity of host cells to G418. If the HCV replicon falls below a threshold level enough to subject hepatocytes to geneticin toxicity, the remaining replicon is beneath detection limit due to decreased cell viability. On the other hand, geneticin selectively amplifies replicon-positive cells above the threshold. The inventors have also carried out rebound studies in the absence of geneticin; however the HCV RNA level in mock- and drug-treated cells all reduced with time due to the lack of selective pressure.

As in HIV management, combination therapy is an important focal point in the development of anti-HCV agents. Optimum combination of drugs with different mechanisms of action should improve efficacy with a wider therapeutic window and reduced viral resistance. It is important that the combination should produce at least additive effects with no antagonism. Our in vitro synergy studies showed that the combination of compound 6 with BILN 2061 or with IFNα-2a was additive to synergistic, more so at the 90% inhibition level (FIG. 6). According to antiviral isobolograms, approximately equipotent combination of compound 6 and BILN 206I (˜0.350EC₉₀) or compound 6 and IFNα-2a (˜0.375EC₉₀) was sufficient to inhibit HCV replication by 90%. The difference between the degree of synergism at the 50% and 90% response level illustrated how the nature of drug-drug interactions may vary depending on the dose ratio in combination and the endpoints of choice (5, 33).

Based on its antiviral specificity and genotypic selectiveness, compound 6 may be targeting the viral replicase, which is supported by observations in our in yilro resistance studies. Whether the binding of octaanionic tetrabiphenylporphyrin to viral protein blocks the interaction with HCV genome, other proteins in the replicase or with host factors is under investigation. If the synthetic agent targets highly conserved sequences that are essential for viral replication (i.e. RNA binding, assembly of replicase), mutations at these hot spots should have decreased probability and as a result it could be difficult for the virus to develop high resistance. While undertaking mechanistic studies, we present here the proof-of-concept design and antiviral results for compound 6, which shows great potential as a potent and selective inhibitor of HCV.

Recent years have seen the rapid advancement of new therapeutic agents against hepatitis C virus (HCV) in response to the need for treatment that is unmet by interferon-based therapies. Most antiviral drugs discovered to date are small molecules that modulate viral enzyme activities. In the search for highly selective protein-binding molecules capable of disrupting viral life cycle, the present inventors have identified a class of anionic tetraphenylporphyrins as potent and specific inhibitors of the HCV replicons. Based on the structure-activity relationship studies reported herein, meso-tetrakis-(3,5-dicarboxy-4,4′-biphenyl)porphyrin was found to be the most potent inhibitor of HCV genotype 1b (Conl) replicon systems but was less effective against genotype 2a (IFH-I) replicon. This compound induced a reduction of viral RNA and protein levels when acting in the low nanomolar range. Moreover the compound could suppress replicon rebound in drug-treated cells and exhibited additive to synergistic effects when combined with protease inhibitor BILN 2061 or with IFNα-2a. The results support and demonstrate the use tetracarboxyphenylporphyrins as potent anti-HCV agents.

### SUPPLEMENTARY EXPERIMENTAL

### Chemical Synthesis and Related Data for Porphyrin Compounds

### Materials

All reagents and solvents were purchased from Aldrich, Fluka, Fisher Scientific, Acros, Mallinckrodt, EM Science, Baker, Strem Chemicals, Novabiochem, or Bachem, unless otherwise stated. All proteins were purchased from Sigma. Silica gel (32-63 μm mesh size) for column chromatography was purchased from Sorbent Technologies. Analytical thin layer chromatography (TLC) was conducted using Baker 0.25 mm silica gel pre-coated glass plates with fluorescent indicator active at UV 245 nm. Preparative TLC was conducted using Analtech 1000 mm silica gel pre-coated glass plates with fluorescent indicator active at UV 245 nm.

### Instruments

Analytical HPLC was conducted on a Ranin HP controller with a Ranin UV detector, both attached to a Dell Optiplex PC running Varian Star Workstation software. Preparative HPLC was conducted on a Waters 600E controller in conjunction with Water 490E multiwavelength UV detector. Both ¹H and ¹³C NMR spectra were obtained on either Bruker DPX 400 or DPX 500 series spectrophotometer at 400 and 100 MHz, or 500 and 125 MHz, respectively. Mass spectrometry data were obtained by Urbana-Champaign Mass Spectrometry Laboratory at University of Illinois.

### 4′-Formyl-biphenyl-4-carboxylic acid ethyl ester

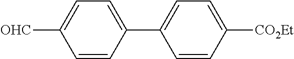

To 4-phenylboronic acid (2.02 g, 13.5 mmol), K₂CO₃ (5.17 g, 37.4 mmol), and Pd(PPh₃)₄ (0.440 g, 0.380 mmol), were added degassed DMF (50 ml) and ethyl 4-bromobenzoate (2.0 ml, 12.2 mmol) under N₂. The mixture was stirred at 120° C. for 27.5 h, and cooled to room temperature. H₂O (100 ml) was added and extracted with diethyl ether (100 ml×4). The collected organic layer was dried over MgSO₄ and the solvent was removed under reduced pressure. The crude product was chromatographed on silica (CHCl₃) to yield 2.89 g (93%) of title compound. m.p. 57-59° C.

¹H NMR (CDCl₃) δ 10.07 (s, 1H), 8.15 (d, J=7.8 Hz, 2H), 7.98 (d, J=7.8 Hz, 2H), 7.78 (d, J=7.8 Hz, 2H), 7.70 (d, J=8.0 Hz, 2H), 4.41 (q, J=7.1 Hz, 2H), 1.42 (t, J=7.1 Hz, 3H).

¹³C NMR (CDCl₃) δ 191.8, 166.2, 145.9, 143.9, 135.7, 130.3, 130.2, 127.9, 127.3, 61.1, 14.3.

HRMS (EI) calcd for C₁₆H₁₄O₃ [M]⁺ 254.09. found 254.09.

### 4′-Formyl-biphenyl-3,5-dicarboxylic acid dimethyl ester

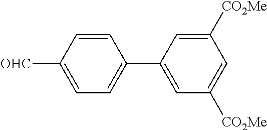

4-Phenylboronic acid (2.00 g, 13.4 mmol), dimethyl 5-bromoisophthalate (3.34 g, 12.2 mmol), K₂CO₃ (5.17 g, 37.4 mmol), and Pd(PPh₃)₄ (0.442 g, 0.381 mmol) were suspended in degassed DMF (50 ml). The mixture was stirred at 100° C. for 18.5 h under N₂, and cooled to room temperature. H₂O (100 ml) was added and extracted with a mixture of diethyl ether (50 ml) and dichloromethane (150 ml). The collected organic layer was dried over Na₂SO₄ and the solvent was removed under reduced pressure. The crude product was recrystallized from EtOH to yield 2.26 g (62%) of title compound. m.p. 166-169° C.

¹H NMR (CDCl₃) δ 10.06 (s, 1H), 8.68 (br t, J=1.5 Hz, 1H), 8.46 (d, J=1.5 Hz, 2H), 7.98 (d, J=8.3 Hz, 2H), 7.80 (d, J=8.3 Hz, 2H), 3.97 (s, 6H).

¹³C NMR (CDCl₃) δ 191.7, 165.8, 140.3, 135.8, 132.3, 132.2, 131.4, 130.3, 127.7, 52.5.

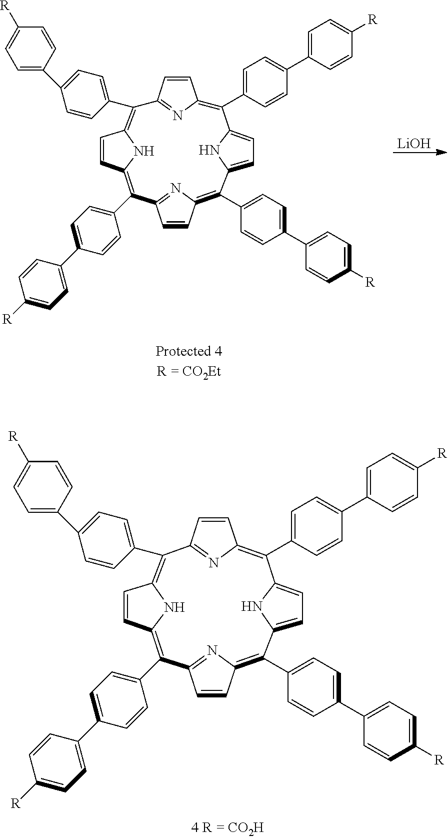

HRMS (EI) calcd for C₁₇H₁₄O₅ [M]⁺ 298.08. found 298.08.

### Tetra-ethylester-tetrabiphenylporphyrin (Protected 4)

Pyrrole (0.175 g, 2.61 mmol), 4′-Formyl-biphenyl-4-carboxylic acid ethyl ester (0.623 g, 2.45 mmol), and Zn(OAc)₂.H₂O (0.139 g, 0.634 mmol) were stirred in acetic acid (12 ml) and refluxed for 1 h. After cooled to room temperature, DDQ (0.328 g, 1.44 mmol) in CHCl₃ (40 ml) was added and stirred overnight. To the mixture silica gel (10g) was added, and stirred at 55° C. for 80 min. The solvent was removed under reduced pressure, and the resulting solid was transferred onto silica gel-packed funnel and washed with a mixture of CHCl₃/AcOEt (10/1). The solvent was removed under reduced pressure, and CHCl₃ (150 ml) and 18% aqueous HCl (150 ml) were added and vigorously stirred for 10 min. The organic layer was collected and washed with saturated NaHCO₃ and dried over Na₂SO₄. The solvent was removed under reduced pressure, and the crude product was chromatographed on silica (CHCl₃/AcOEt=20/1) and Sephadex LH-20 (CH₂Cl₂/AcOEt=5/1) to yield 81.8 mg (11%) or Protected 4.

m.p. >340° C. ¹H NMR (CDCl₃) δ 8.91 (s, 8H), 8.25 (ap t, J=8.7 Hz, 16H), 7.93 (d, J=7.8 Hz, 16H), 4.49 (q, J=7.1 Hz, 8H), 1.49 (t, J=7.1 Hz, 12H), −2.65 (br s, 2H).

¹³C NMR (CDCl₃) δ 166.5, 144.9, 141.8, 139.2, 135.1, 130.2, 129.5, 127.1, 125.5, 119.7, 61.1, 14.4. HRMS (FAB) calcd for C₈₀H₆₃N₄O₈ [M+H]⁺ 1207.46. found 1207.46.

### Tetracarboxybiphenylporphyrin (Compound 4)

Protected 4 (82 mg, 0.0679 mmol) was dissolved in 1,4-dioxane (64 ml) MeOH (16 ml), and 1 N LiOH (4.0 ml), and stirred at room temperature for 21 h. The solvent was removed under reduced pressure and the resulting solid was washed with 1,4-dioxane. The solid was dissolved in H₂O and acidified with 36% aqueous HCl. The suspension was centrifuged and the supernatant was removed. The wash with dilute aqueous HCl solution was repeated and the product was lyophilized to yield 80.1 mg (quant.) of 4 as HCl salt. m.p. >340° C.

¹H NMR (DMSO-d₆) δ 8.97 (s, 8H), 8.38 (d, J=8.1 Hz, 8H), 8.24 (d, J=8.3 Hz, 8H), 8.18 (ap s, 16H). MALDI-TOF MS calcd for C₇₂H₄₈N₂O₈ [M+2H]⁺ 1096.35. found 1096.44.

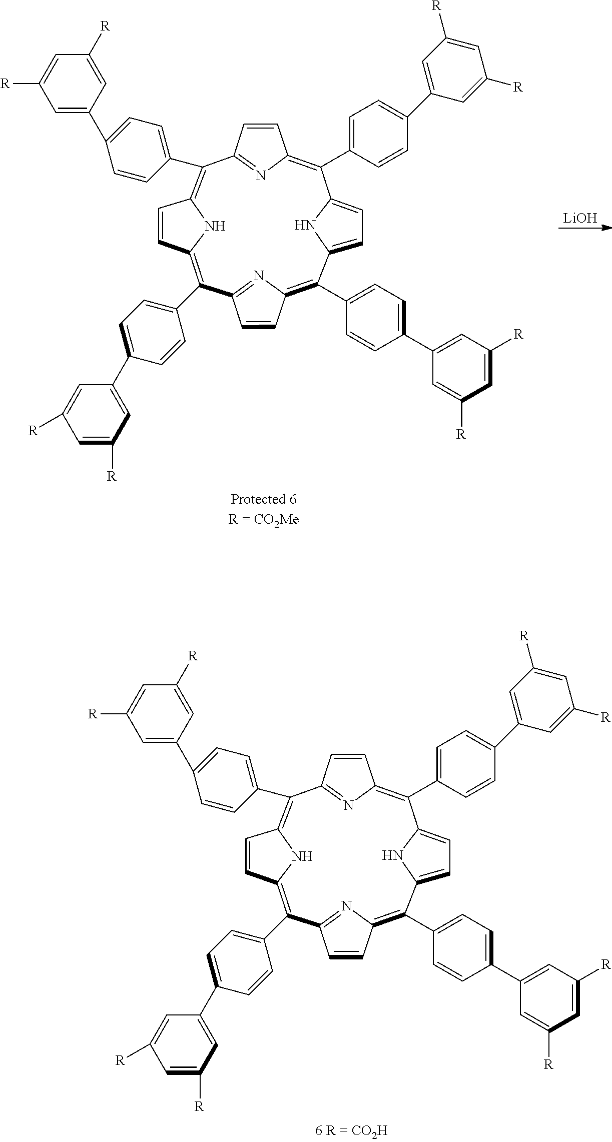

### Octa-methylester-tetrabiphenylporphyrin (Protected 6)

Pyrrole (0.346 g, 5.15 mmol), 4′-Formyl-biphenyl-3,5-dicarboxylic acid dimethyl ester (1.54 g, 5.15 mmol), and Zn(OAc)₂.H₂O (0.289 g, 1.31 mmol) were stirred in acetic acid (25 ml) and refluxed for 1.5 h. The solvent was removed under reduced pressure, and DDQ (0.575 g, 2.53 mmol) in CHCl₃ (40 ml) was added and stirred for 1 h. To the mixture silica gel (20 g) was added, and stirred at 50° C. for 1 h. The mixture was passed through silica gel-packed funnel and washed with CHCl₃/AcOEt (10/1). The solvent was removed under reduced pressure, and CH₂Cl₂ (200 ml) and 18% aqueous HCl (200 ml) were added and vigorously stirred for 10 min. The organic layer was collected and washed with saturated NaHCO₃ and dried over Na₂SO₄. The solvent was removed under reduced pressure, and the crude product was chromatographed on silica (CH₂Cl₂/AcOEt=20/1) to yield 293 mg (16%) or protected 6.

m.p. >340° C. ¹H NMR (CDCl₃) δ 8.96 (s, 8H), 8.82 (d, J=1.5 Hz, 8H), 8.80 (t, J=1.5 Hz, 4H), 8.37 (d, J=8.1 Hz, 8H), 8.09 (d, J=8.1 Hz, 8H), 4.07 (s, 24H), −2.68 (br s, 2H).

¹³C NMR (CDCl₃) δ 166.3, 142.1, 141.6, 138.4, 135.2, 132.5, 131.4, 129.7, 125.6, 119.6, 52.6. HRMS (FAB) calcd for C₈₄H₆₃N₄O₁₆ [M+H]⁺ 1383.42. found 1383.42.

### Octa-carboxy-tetrabiphenylporphyrin (Compound 6)

Protected 6 (85 mg, 0.0614 mmol) was dissolved in 1,4-dioxane (64 ml), MeOH (16 ml), and 1 N LiOH (4.0 ml), and stirred at room temperature for 24 h. The solvent was removed under reduced pressure and the resulting solid was washed with 1,4-dioxane. The solid was redissolved in MeOH (30 ml), H₂O (45 ml), and 1 N LiOH (5.0 ml), and stirred at room temperature for 21 h. The solvent was removed under reduced pressure, and redissolved in H₂O and acidified with 36% aqueous HCl. The suspension was centrifuged and the supernatant was removed. The wash with dilute aqueous HCl solution was repeated and the product was lyophilized to yield 70.6 mg (86%) of 6 as HCl salt.

m.p. >340° C. ¹H NMR (DMSO-d₆) δ 8.97 (s, 8H), 8.73 (s, 8H), 8.60 (s, 4H), 8.37 (d, J=7.8 Hz, 8H), 8.21 (d, J=7.8 Hz, 8H).

¹³C NMR (DMSO-d₆) δ 166.5, 141.1, 140.6, 137.9, 135.1, 132.3, 131.6, 125.5, 119.5.

MALDI-TOF MS calcd for C₇₂H₄₈N₂O₈ [M+2H]⁺ 1272.31. found 1271.90.

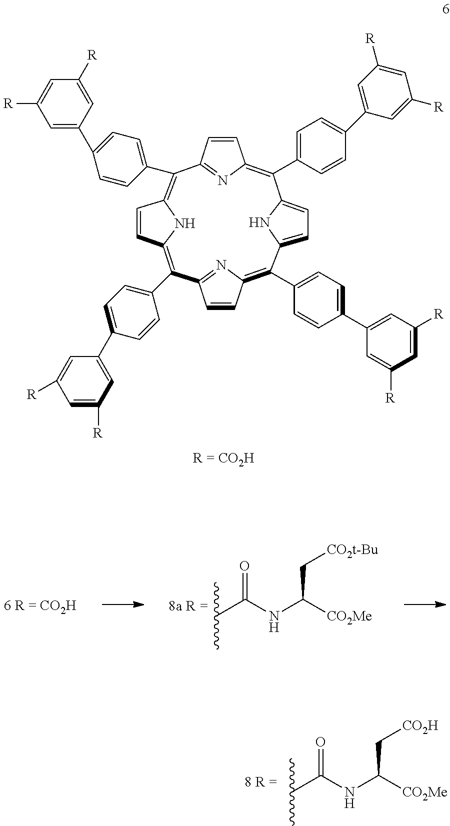

### Tetrabiphenylporphyrin (8a)

To a suspension of 6 (20.3 mg, 0.0151 mmol) in dry CH₂Cl₂ (3.5 ml), oxalyl chloride (0.55 ml, 6.30 mmol), was added DMF (40 μl, 0.517 mmol) and stirred under N₂ for 17 h. The solvent was removed under reduced pressure and dried in vacuo to yield dark green solid. The solid was redissolved in dry THF (3.5 ml), and H-Asp(Ot-Bu)-OMe.HCl (59.5 mg, 0.248 mmol) in dry CH₂Cl₂ (3.5 ml) and diisopropylethylamine (0.30 ml) was added. The solution was stirred under N₂ for 4 h before CH₂Cl₂ (60 ml) was added. The solution was washed with H₂O, 0.5 N HCl, saturated NaHCO₃, and brine, and dried over Na₂SO₄. The solvent was removed under reduced pressure, and the crude product was chromatographed on silica (CH₂Cl₂/MeOH=5/1) to yield 28.2 mg (68%) of 8a.

m.p. 130-170° C. (very viscous so difficult to determine exact m.p.).

¹H NMR (CDCl₃) δ 8.97 (s, 8H), 8.52 (d, J=1.5 Hz, 8H), 8.38 (d, J=8.3 Hz, 8H), 8.33 (t, J=1.5 Hz, 4H), 8.07 (d, J=8.3 Hz, 8H), 7.46 (d, J=8.1 Hz, 8H), 5.17-5.12 (m, 4H), 3.85 (s, 12H), 3.12 (dd, J=16.9, 4.5 Hz, 8H), 2.96 (dd, J=16.9, 4.5 Hz, 8H), 1.49 (s, 72H), −2.68 (br s, 2H).

MALDI-TOF MS calcd for C₁₄₈ H₁₇₀N₁₂O₄₀ [M+4H]⁺ 2755.16. found 2754.91.

### Tetrabiphenylporphyrin (Compound 8)

8a (9.63 mg, 0.00524 mmol) was dissolved in TFA (7.6 ml) and H₂O (0.40 ml), and stirred for 4 h at room temperature. The solvent was removed under reduced pressure, and the crude product was purified by HPLC (RP-C₁₈: Gradient of 10% acetonitrile linearly increasing to 90% over 40 min., in 0.1% TFA/H₂O) and lyophilized to yield 5.65 mg (59%) of 3 as TFA salt. m.p. >340° C.

¹H NMR (DMSO-d₆) δ 9.06 (d, J=7.6 Hz, 4H), 8.98 (s, 8H), 8.39 (d, J=8.2 Hz, 8H), 8.25 (d, J=8.5 Hz, 8H), 8.18 (d, J=8.2 Hz, 8H), 8.12 (d, J=8.5 Hz, 8H), 4.91-4.87 (m, 4H), 3.70 (s, 12H), 2.94 (dd, J=16.1, 5.4 Hz, 4H), 2.82 (dd, J=16.4, 7.9 Hz, 4H), −2.82 (br s, 2H).

MALDI-TOF MS calcd for C₉₂H₇₆N₈O₂₀ [M+2H]⁺ 1556.45. found 1557.46.

### Tetrabiphenylporphyrin (Compounds 2, 5 and 7)

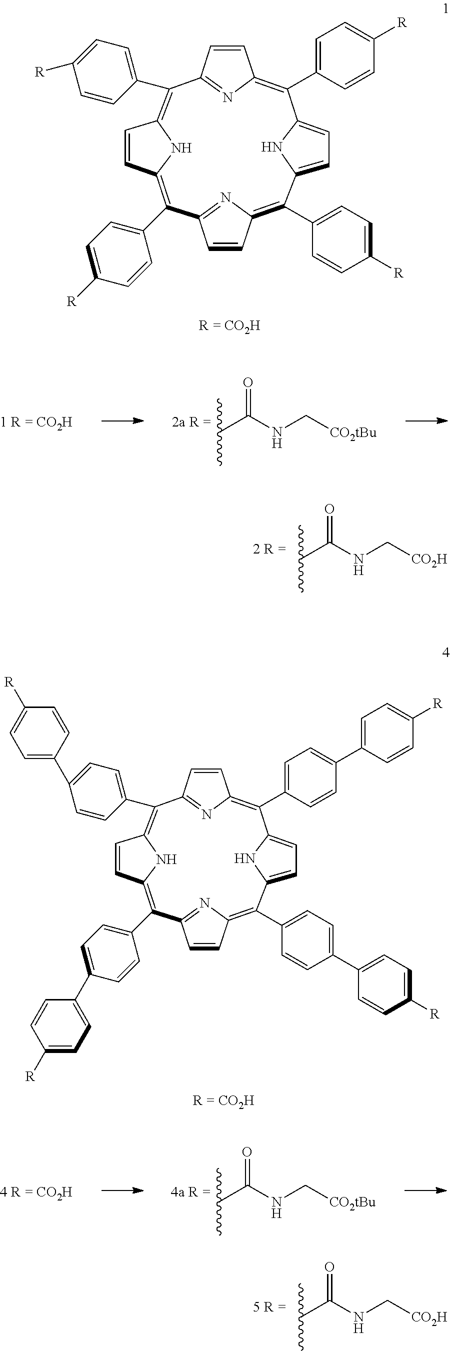

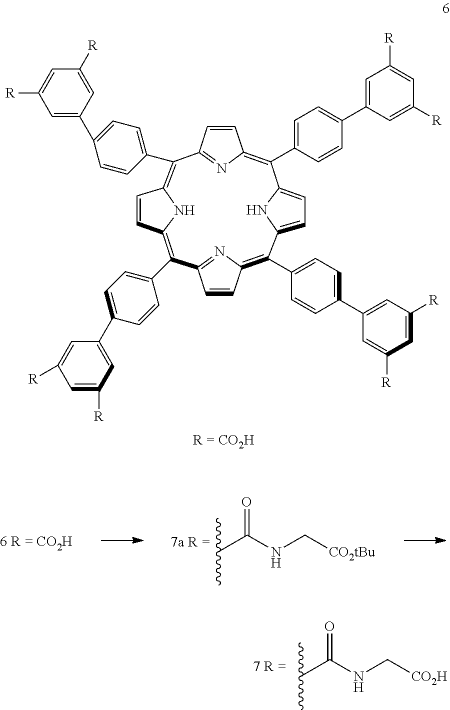

Compounds 2, 5 and 7 were synthesized following similar synthetic procedures of compound 8. H-Gly-OtBu-HCl was used as the amino acid choice for compounds 2, 5 and 7.

Compound 2: yield: 44% over 2 steps. ¹H NMR (DMSO-d₆) δ 9.03 (t, J=5.8 Hz, 4H), 8.86 (s, 8H), 8.38-8.33 (m, 16H), 3.69 (q, J=6.0 Hz, 8H), −2.91 (s, b, 2H).

MALDI-TOF MS calcd for C₅₆H₄₂N₈O₁₂ [M+H]⁺ 1018.29. found 1018.44.

Compound 5: yield: 35% over 2 steps. ¹H NMR (DMSO-d₆) δ 8.97 (s, 8H), 8.64 (t, J=5.8 Hz, 4H), 8.38 (d, J=8.1 Hz, 8H), 8.24 (d, J=8.3 Hz, 8H), 8.18 (ap s, 16H), 3.72 (q, J=6.0 Hz, 8H).

MALDI-TOF MS calcd for C₈₀H₅₈N₈O₁₂ [M+H]⁺ 1323.42. found 1323.74.

Compound 7: yield: 28% over 2 steps. ¹H NMR (DMSO-d₆) δ 8.99 (s, 8H), 8.81 (t, J=5.8 Hz, 8H), 8.73 (s, 8H), 8.60 (s, 4H), 8.37 (d, J=7.8 Hz, 8H), 8.21 (d, J=7.8 Hz, 8H), 3.65 (m, 16H).

MALDI-TOF MS calcd for C₉₂H₇₀N₁₂O₂₄ [M+H]⁺ 1727.46. found 1727.28.

TABLE 1

| | 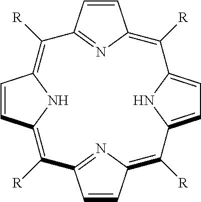 | | | | | | | |
|---|---|---|---|---|---|---|---|---|
| | | | | | | | | Mitochondrial |
| | | Antiviral Activity | | | Cytotoxicity | | | DNA Toxicity |
| | | EC₅₀ (μM) | | | CC₅₀ (μM) | | | IC₅₀ (μM) |
| # | R-Group | HCV | HBV | HIV-1 IIIB | MT-2 | CEM | Huh-7 | CEM |
| 1 | 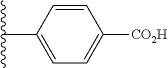 | 0.243 ± 0.0359 | >10 | >25 | >25 | 34 | >50 | >50 |
| 2 | 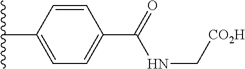 | 1.8 ± 0.70 | >10 | >25 (>CC₅₀) | 3.2 ± 1.13 | 27 ± 15.13 | >50 | 15 ± 3.7 |
| 3 | 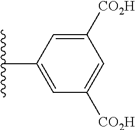 | 13.6 ± 1.10 | 7 | >100 (>CC₅₀) | 22 | >50 | >50 | 25 |
| 4 | 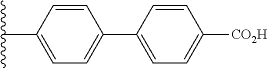 | 0.877 ± 0.0289 | >10 | >25 | >25 | 10 | 32.2 ± 4.14 | 6 |
| 5 | 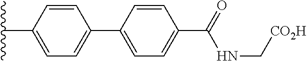 | 0.148 ± 0.0060 | >20 | 1.50 ± 0.436 | 13.0 ± 4.36 | >50 | >50 | >50 |
| 6 | 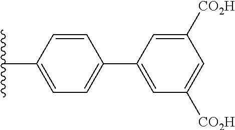 | 0.024 ± 0.0051 | >20 | 3.67 ± 1.222 | 18.0 ± 4.24 | >50 | >50 | >50 |
| 7 | 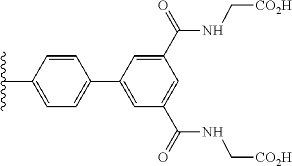 | >12.5 | >10 | 1.05 ± 0.071 | 15.0 ± 4.24 | >50 | 12.5 | 12.5 |
| 8 | 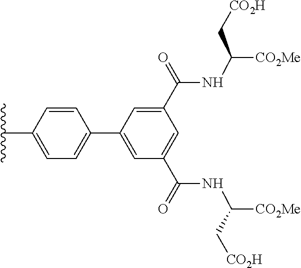 | 2.5 ± 0.20 | >10 | 4.10 ± 1.556 | >25 | * | 12.5 | * |
| 9 | 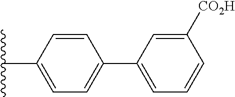 | 0.764 ± 0.0600 | 8 | >25 (>CC₅₀) | >25 | 50 | 13.0 ± 0.70 | 22 |

TABLE 2
Effect of serum concentration on the EC₅₀s of compounds 1, 4 & 6
A.

| | EC₅₀ (μM) | | | | |
|---|---|---|---|---|---|
| | 0% FBS | | | | |
| Compound | (Theoretical) | 5% FBS | 10% FBS | 20% FBS | 40% FBS |
| 1 | 0.0482 | 0.124 ± 0.008 | 0.243 ± 0.036 | 0.513 ± 0.069 | 0.948 ± 0.089 |
| 4 | 0.3800 | 0.633 ± 0.176 | 0.877 ± 0.029 | 1.210 ± 0.056 | 1.483 ± 0.015 |
| 6 | 0.0065 | 0.013 ± 0.003 | 0.024 ± 0.005 | 0.039 ± 0.005 | 0.069 ± 0.011 |

B.

Fold Change in EC₅₀

| Compound | 5% FBS | 10% FBS | 20% FBS | 40% FBS |
|---|---|---|---|---|
| 1 | 1.00 | 1.95 ± 0.16 | 4.16 ± 0.82 | 7.62 ± 0.46 |
| 4 | 1.00 | 1.46 ± 0.42 | 2.01 ± 0.52 | 2.48 ± 0.74 |
| 6 | 1.00 | 1.92 ± 0.22 | 3.20 ± 0.96 | 5.56 ± 0.85 |

TABLE 3
Metallation of porphyrin core did not significantly affect the anti-HCV activity of compound 6

6

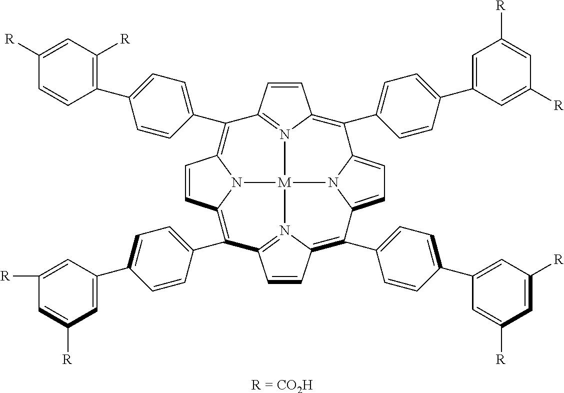

| Metal (M) | EC₅₀ (μM) |
|---|---|
| — | 0.019 ± 0.0052 |
| Zn (II) | 0.017 ± 0.0071 |
| Cu (II) | 0.025 ± 0.0029 |
| Fe (II) | 0.019 ± 0.0067 |

### REFERENCES

1. Aya, T., and A. D. Hamilton. 2003. Tetrabiphenylporphyrin-based receptors for protein surfaces show sub-nanomolar affinity and enhance unfolding. Bioorg. Med. Chem. Lett. 13:2651-2654.
2. Blight, K. J., A. A. Kolykhalov, and C. M. Rice. 2000. Efficient initiation of HCV RNA replication in cell culture. Science 290:1972-1974.
3. Chen-Collins, A. R., D. W. Dixon, A. N. Vzorov, L. G. Marzilli, and R. W. Compans. 2003. Prevention of poxvirus infection by tetrapyrroles. BMC Infect. Dis. 3:9.
4. Choo, Q. L., K. H. Richman, J. H. Han, K. Berger, C. Lee, C. Dong, C. Gallegos, D. Coit, R. Medina-Selby, and P. J. Barr. 1991. Genetic organization and diversity of the hepatitis C virus. Proc. Natl. Acad. Sci. U.S.A. 88:2451-2455.
5. Chou, T.-C. 1991. The median-effect principle and the combination index for quantitation of synergism and antagonism, p. 61-102. In D. C. Rideout and T.-C. Chou (ed.), Synergism and antagonism in chemotherapy. Academic Press, Inc., San Diego, Calif.
6. De Francesco, R., and G. Migliaccio. 2005 Challenges and successes in developing new therapies for hepatitis C. Nature 436:953-960.
7. Dixon, D. W., A. F. Gill, L. Giribabu, A. N. Vzorov, A. B. Alam, and R. W. Compans. 2005. Sulfonated naphthyl porphyrins as agents against HIV-1. J. Inorg. Biochem. 99:813-821.
8. El-Hage, N., and G. Luo. 2003. Replication of hepatitis C virus RNA occurs in a membrane-bound replication complex containing nonstructural viral proteins and RNA. J. Gen. Virol. 84:2761-2769.
9. Fletcher, S., and A. D. Hamilton. 2005. Protein surface recognition and proteomimetics: mimics of protein surface structure and function. Curr. Opin. Chem. Biol. 9:632-638.
10. Foy, E., K. Li, C. Wang, R. Sumpter, Jr., M. Ikeda, S. M. Lemon, and M. Gale, Jr. 2003. Regulation of interferon regulatory factor-3 by the hepatitis C virus serine protease. Science 300:1145-1148.
11. Fried, M. W., M. L. Shiffman, K. R. Reddy, C. Smith, G. Marinos, F. L. Goncales, Jr., D. Haussinger, M. Diago, G. Carosi, D. Dhumeaux, A. Craxi, A. Lin, J. Hoffman, and J. Yu. 2002. Peginterferon alfa-2a plus ribavirin for chronic hepatitis C virus infection. N. Engl. I Med. 347:975-982.
12. Gradl, S, N., J. P. Felix, E. Y. Isacoff, M. L. Garcia, and D. Trauner. 2003. Protein surface recognition by rational design: nanomolar ligands for potassium channels. J. Am. Chem. Soc. 125:12668-12669.
13. Greco, W. R., H. S. Park, and Y. M. Rustum. 1990. Application of a new approach for the quantitation of drug synergism to the combination of cis-diamminedichloroplatinum and 1-beta-D-arabinofuranosylcytosine. Cancer Res. 50:5318-5327.
14. Hardy, R. W., J. Marcotrigiano, K. J. Blight, J. E. Majors, and C. M. Rice. 2003. Hepatitis C virus RNA synthesis in a cell-free system isolated from replicon-containing hepatoma cells. J. Virol. 77:2029-2037.
15. Hoofnagle, J. H. 1997. Hepatitis C: the clinical spectrum of disease. Hepatology 26:15 S-20S.
16. Jain, R. K., and A. D. Hamilton. 2000. Protein surface recognition by synthetic receptors based on a tetraphenylporphyrin scaffold. Org. Lett. 2:1721-1723.
17. Lam, W., C. Chen, S. Ruan, C. H. Leung, and Y. C. Cheng. 2005. Expression of deoxynucleotide carrier is not associated with the mitochondrial DNA depletion caused by anti-HIV dideoxynucleoside analogs and mitochondrial dNTP uptake. Mol. Pharmacol. 67:408-416.
18. Lamarre, D., P. C. Anderson, M. Bailey, P. Beaulieu, G. Bolger, P. Bonneau, M. Bos, D. R. Cameron, M. Cartier, M. G. Cordingley, A. M. Faucher, N. Goudreau, S. H. Kawai, G. Kukolj, L. Lagace, S. R. LaPlante, H. Narjes, M. A. Poupart, J. Rancourt, R. E. Sentjens, R. St. George, B. Simoneau, G. Steinmann, D. Thibeault, Y. S. Tsantrizos, S. M. Weldon, C. L. Yong, and M. Llinas-Brunet. 2003. An NS3 protease inhibitor with antiviral effects in humans infected with hepatitis C virus. Nature 426:186-189.
19. Leung, C. H., S. P. Grill, W. Lam, Q. B. Han, H. D. Sun, and Y. C. Cheng. 2005. Novel mechanism of inhibition of nuclear factor-kappa B DNA-binding activity by diterpenoids isolated from Isodon rubescens. Mol. Pharmacol. 68:286-297.
20. Lindahl, K., L. Stahle, A. Bruchfeld, and R. Schvarcz. 2005. High-dose ribavirin in combination with standard dose peginterferon for treatment of patients with chronic hepatitis C. Hepatology 41:275-279.
21. Lindenbach, B. D., M. J. Evans, A. J. Syder, B. Wolk, T. L. Tellinghuisen, C. C. Liu, T. Maruyama, R. O. Hynes, D. R. Burton, J. A. McKeating, and C. M. Rice. 2005. Complete replication of hepatitis C virus in cell culture. Science 309:623-626.
22. Lindenbach, B. D., B. M. Pragai, R. Montserret, R. K. Beran, A. M. Pyle, F. Penin, and C. M. Rice. 2007. The C terminus of hepatitis C virus NS4A encodes an electrostatic switch that regulates NS5A hyperphosphorylation and viral replication. J. Virol. 81:8905-8918.
23. Lohmann, V., F. Ko\ddot{}rner, J. O. Koch, U. Herian, L. Theilmann, and R. Bartenschlager. 1999. Replication of subgenomic hepatitis C virus RNAs in a hepatoma cell line. Science 285:110-113.
24. Manns, M. P., G. R. Foster, J. K. Rockstroh, S. Zeuzem, F. Zoulim, and M. Houghton. 2007. The way forward in HCV treatment—finding the right path. Nat. Rev. Drug Discov. 6:991-1000.
25. Manns, M. P., J. G. McHutchison, S. C. Gordon, V. K. Rustgi, M. Shiffman, R. Reindollar, Z. D. Goodman, K. Koury, M.-H. Ling, and J. K. Albrecht. 2001. Peginterferon alfa-2b plus ribavirin compared with interferon alfa-2b plus ribavirin for initial treatment of chronic hepatitis C: a randomised trial. Lancet 358:958-965.
26. Mellors, J. W., G. E. Dutschman, G. J. Im, E. Tramontano, S. R. Winkler, and Y. C. Cheng. 1992. In vitro selection and molecular characterization of human immunodeficiency virus-1 resistant to non-nucleoside inhibitors of reverse transcriptase. Mol. Pharmacol. 41:446-451.
27. Miyamoto, M., T. Kato, T. Date, M. Mizokami, and T. Wakita. 2006. Comparison between subgenomic replicons of hepatitis C virus genotypes 2a (JFH-1) and 1b (Con1 NK5.1). Intervirology 49:37-43.
28, Pereira, A. A., and I. M. Jacobson. 2009. New and experimental therapies for HCV. Nat. Rev. Gastroenterol. Hepatol. 6:403-411.
29. Pflugheber, J., B. Fredericksen, R. Sumpter, Jr., C. Wang, F. Ware, D. L. Sodora, and M. Gale, Jr. 2002. Regulation of PKR and IRF-1 during hepatitis C virus RNA replication. Proc. Natl. Acad. Sci. U.S.A. 99:4650-4655.
30. Phan, T., R. K. Beran, C. Peters, I. Lorenz, and B. D. Lindenbach. 2009. Hepatitis C virus NS2 protein contributes to virus particle assembly via opposing epistatic interactions with the E1-E2 glycoprotein and NS3-NS4A enzyme complexes. J. Virol. 83:8379-8395.
31. Quinkert, D., R. Bartenschlager, and V. Lohmann. 2005. Quantitative analysis of the hepatitis C virus replication complex. J. Virol. 79:13594-13605.
32. Rideout, D. C. 1991. The median-effect principle and the combination index for quantitation of synergism and antagonism, p. 3-60. In D. C. Rideout and T.-C. Chou (ed.), Synergism and antagonism in chemotherapy. Academic Press, Inc., San Diego, Calif.
33. Simmonds, P., J. Bukh, C. Combet, G. Dele'age, N. Enomoto, S. Feinstone, P. Halfon, G. Inchauspe', C. Kuiken, G. Maertens, M. Mizokami, D. G. Murphy, H. Okamoto, J.-M. Pawlotsky, F. Penin, E. Sablon, T. Shin-I, L. J. Stuyver, H.-J. Thiel, S. Viazov, A. J. Weiner, and A. Widell. 2005. Consensus proposals for a unified system of nomenclature of hepatitis C virus genotypes. Hepatology 42:962-973.
34. Stites, W. E. 1997. Protein-protein interactions: interface structure, binding thermodynamics, and mutational analysis. Chem. Rev. 97:1233-1250.
35. Trozzi, C., L. Bartholomew, A. Ceccacci, G. Biasiol, L. Pacini, S. Altamura, F. Narjes, E. Muraglia, G. Paonessa, U. Koch, R. De Francesco, C. Steinkuhler, and G. Migliaccio. 2003. In vitro selection and characterization of hepatitis C virus serine protease variants resistant to an active-site peptide inhibitor. J. Virol. 77:3669-3679.
36. Tsantrizos, Y. S. 2004. The design of a potent inhibitor of the hepatitis C virus NS3 protease: BILN 2061—from the NMR tube to the clinic. Biopolymers 76:309-323.
37. Vrolijk, J. M., A. Kaul, B. E. Hansen, V. Lohmann, B. L. Haagmans, S. W. Schalm, and R. Bartenschlager. 2003. A replicon-based bioassay for the measurement of interferons in patients with chronic hepatitis C. J. Virol. Methods 110:201-209.
38. Vzorov, A. N., D. W. Dixon, J. S. Trommel, L. G. Marzilli, and R. W. Compans. 2002. Inactivation of human immunodeficiency virus type 1 by porphyrins. Antimicrob. Agents Chemother. 46:3917-3925.
39. Wasley, A., and M. J. Alter. 2000. Epidemiology of hepatitis C: geographic differences and temporal trends. Semin. Liver Dis. 20:1-16.
40. Yang, W., Y. Zhao, J. Fabrycki, X. Hou, X. Nie, A. Sanchez, A. Phadke, M. Deshpande, A. Agarwal, and M. Huang. 2008. Selection of replicon variants resistant to ACH-806, a novel hepatitis C virus inhibitor with no crossresistance to NS3 protease and NS5B polymerase inhibitors. Antimicrob. Agents Chemother. 52:2043-2052.
41. Yeo, H., Y. Li, L. Fu, J. L. Zhu, E. A. Gullen, G. E. Dutschman, Y. Lee, R. Chung, E. S. Huang, D. J. Austin, and Y. C. Cheng. 2005. Synthesis and antiviral activity of helioxanthin analogues. J. Med. Chem. 48:534-546.

1. A compound according to the chemical formula: 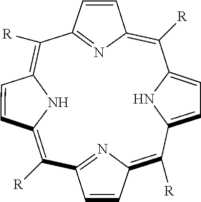
Wherein each R group is independently a substituted phenyl group, wherein said phenyl group is substituted with at least one carboxylic acid group(s) or at least one group containing a carboxylic acid group, or a biphenyl group which is substituted on the distil phenyl group with 1, 2 or 3 carboxylic acid group(s) or at least one and up to three groups containing a carboxylic acid group, with the proviso that when R is a phenyl group, said phenyl group is substituted with at least one group other than a single carboxylic acid group, or a pharmaceutically acceptable salt, solvate or polymorph thereof, optionally in combination with a metal.

2. The compound according to claim 1 wherein said metal is selected from the group consisting of Fe III (Fe3+), Fe II, Cu II, Zn II, Mg II and Mn II.

3. The compound according to claim 1 wherein each R is identical.

4. The compound according to claim 1 wherein R is a —X—(CH₂)ₙCOOH group, a
—X—(CH₂O)ⱼCOOH group, a —X—(CH₂CHYO)ₖCOOH group, a C(O)—NZ—(CH₂)ₘCOOH group, a 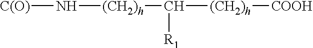
group, an optionally substituted biphenyl group wherein at least the distil phenyl contains at least one and up to three R′ group(s), where R′ is a —X—(CH₂)_{n′}COOH group, a —X—(CH₂O)ⱼCOOH group, a —X—(CH₂CHYO)ₖCOOH group, a C(O)—NZ—(CH₂)ₘ—COOH group or a 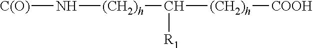
group;
Where R₁ is an amino acid sidechain from alanine, arginine, asparagine, aspartic acid, cysteine, glutamine, glutamic acid, glycine, histidine, isoleucine, leucine, lysine, methionine, norleucine, phenylalanine, proline, serine, threonine, tryptophan, tyrosine or valine, or R₁ and the adjacent nitrogen atom form a cyclic sidechain from proline;
X is absent, O, S or N—Z;
Y is H or CH₃;
Z is H or a C₁-C₃ alkyl group;
each h is independently 0 to 2;
j is an integer from 0 to 10;
k is an integer from 0 to 6;
m is an integer from 0 to 10;
n is an integer from 0 to 12;
and n′ is an integer from 0 to 12; or
a pharmaceutically acceptable salt, solvate or polymorph thereof, with the proviso that when each R in said compound is identical and is a phenyl group substituted with only one group, that group is other than a carboxylic acid group.

5. The compound according to claim 4 wherein R₁ is a sidechain from alanine, arginine, asparagine, aspartic acid, cysteine, glutamine, glutamic acid, glycine, histidine, isoleucine, leucine, lysine, methionine, norleucine, phenylalanine, serine, threonine, tryptophan, tyrosine or valine, or R₁ and the adjacent nitrogen atom form a cyclic sidechain from proline.

6. The compound according to claim 5 wherein R₁ is a sidechain from aspartic acid or glutamic acid and one of the two carboxylic acid groups in the sidechain is optionally esterified with a C₁-C₆ alkyl group.

7. The compound according to claim 4 wherein R₁ is H, C₁-C₄ alkyl, CH₂OH, C₂-C₄ thioether, benzyl or p-hydroxybenzyl.

8. The compound according to claim 1 wherein R is a biphenyl group substituted with two carboxylic acid groups at meta positions on the distal phenyl group of the biphenyl group, or a pharmaceutically acceptable salt thereof.

9. A pharmaceutical composition comprising an effective amount of a compound according to the formula: 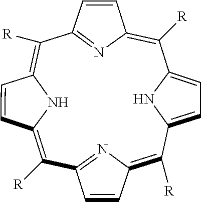
Wherein each R group is independently a substituted phenyl group, wherein said phenyl group is substituted with at least one carboxylic acid group(s) or at least one group containing a carboxylic acid group, or a biphenyl group which is substituted on the distil phenyl group with 1, 2 or 3 carboxylic acid group(s) or at least one and up to three groups containing a carboxylic acid group, with the proviso that when R is a phenyl group, said phenyl group is substituted with at least one group other than a single carboxylic acid group, or a pharmaceutically acceptable salt, solvate or polymorph thereof in combination with a pharmaceutically acceptable carrier, additive or excipient, optionally in combination with a metal.

10. The composition according claim 9 wherein said metal is selected from the group consisting of Fe III (Fe3+), Fe II, Cu II, Zn II, Mg II and Mn II.

11. The composition according to claim 9 wherein each R is identical.

12. The composition according to claim 9 wherein R is a —X—(CH₂)ₙCOOH group, a
—X—(CH₂O)ⱼCOOH group, a —X—(CH₂CHYO)ₖCOOH group, a C(O)—NZ—(CH₂)ₘCOOH group, a 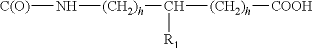
group, an optionally substituted biphenyl group wherein at least the distil phenyl contains at least one and up to three R′ group(s), where R′ is a —X—(CH₂)_{n′}COOH group, a —X—(CH₂O)ⱼCOOH group, a —X—(CH₂CHYO)ₖCOOH group, a C(O)—NZ—(CH₂)ₘCOOH group or a 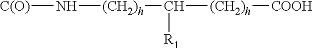
group;
Where R₁ is an amino acid sidechain from alanine, arginine, asparagine, aspartic acid, cysteine, glutamine, glutamic acid, glycine, histidine, isoleucine, leucine, lysine, methionine, norleucine, phenylalanine, proline, serine, threonine, tryptophan, tyrosine or valine, or R₁ and the adjacent nitrogen atom form a cyclic sidechain from proline;
X is absent, O, S or N—Z;
Y is H or CH₃;
Z is H or a C₁-C₃ alkyl group;
each h is independently 0 to 2;
j is an integer from 0 to 10;
k is an integer from 0 to 6;
m is an integer from 0 to 10;
n is an integer from 0 to 12;
and n′ is an integer from 0 to 12; or
a pharmaceutically acceptable salt, solvate or polymorph thereof.

13. The composition according to claim 12 wherein R₁ is a sidechain from alanine, arginine, asparagine, aspartic acid, cysteine, glutamine, glutamic acid, glycine, histidine, isoleucine, leucine, lysine, methionine, norleucine, phenylalanine, serine, threonine, tryptophan, tyrosine or valine, or R₁ and the adjacent nitrogen atom form a cyclic sidechain from proline.

14. The composition according to claim 13 wherein R₁ is a sidechain from aspartic acid or glutamic acid and one of the two carboxylic acid groups in the sidechain is optionally esterified with a C₁-C₆ alkyl group.

15. The composition according to claim 12 wherein R₁ is H, C₁-C₄ alkyl, CH₂OH, C₂-C₄ thioether, benzyl or p-hydroxybenzyl.

16. The composition according to claim 9 wherein R is a biphenyl group substituted with two carboxylic acid groups at meta positions on the distal phenyl group of the biphenyl group, or a pharmaceutically acceptable salt thereof.

17. The composition according to claim 12 further comprising at least one additional anti-HCV agent.

18. The composition according to claim 17 wherein said additional anti-HCV agent is selected from the group consisting of interferon (IFN), ribavirin or a mixture thereof.

19. The composition according claim 17 further including a compound selected from the group consisting of BILN 2061, G418, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, ACH-1095, GSK625433, TG4040 (MVA-HCV), A-831, F351, NS5A, NS4B, ANA598, A-689, GNI-104, IDX102, ADX184, GL59728, GL60667, PSI-7851, TLR9Agonist, PHX1766, SP-30, VCH-222 and mixtures thereof.

20. The composition according to claim 12 further comprising an anti-cancer agent.

21. The composition according to claim 20 wherein said anti-cancer agent is selected from the group consisting of doxorubicin (adriamycin), cis platin and mixtures thereof.

22. A compound according to the formula: 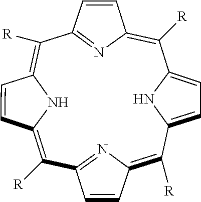
Wherein at least one of R is a 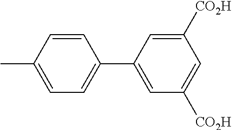
group or a pharmaceutically acceptable salt thereof.

23. The compound according to claim 22 when each R is identical.

24. A pharmaceutical composition comprising an effective amount of a compound according to claims 22 in combination with a carrier, additive or excipient.

25. The composition according to claim 24 further comprising at least one additional anti-HCV agent.

26. The composition according to claim 24 further comprising an anti-cancer agent.

27. The composition according to claim 26 wherein said anti-cancer agent is doxorubicin, cis platin or mixtures thereof.

28. A method of treating a flaviviridae virus infection in a patient or subject in need thereof comprising administering to said patient or subject an effective amount of a pharmaceutical composition according to claim 9.

29. The method according to claim 28 wherein said virus infection is Hepatitis C virus (HCV), bovine viral diarrhea virus (BVDV), hog cholera (swine fever), yellow fever and West Nile virus.

30. The method according to claim 28 wherein said virus infection is HCV.

31. A method of inhibiting a flaviviridae virus infection in a patient or subject in need thereof comprising administering to said patient or subject an effective amount of a pharmaceutical composition according to claim 9.

32. The method according to claim 31 wherein said virus infection is Hepatitis C virus (HCV), bovine viral diarrhea virus (BVDV), hog cholera (swine fever), yellow fever and West Nile virus.

33. The method according to claim 32 wherein said virus infection is HCV.

34. A method of reducing the likelihood of a flaviviridae virus infection in a patient or subject at risk for such an infection comprising administering to said patient or subject an effective amount of a pharmaceutical composition according to claim 9.

35. The method according to claim 34 wherein said virus infection is Hepatitis C virus (HCV), bovine viral diarrhea virus (BVDV), hog cholera (swine fever), yellow fever and West Nile virus.

36. The method according to claim 34 wherein said virus infection is HCV.

37. A method of reducing the likelihood of a relapse of an HCV infection in a patient or subject who has been cured of HCV, said method comprising administering to said patient or subject an effective amount of a pharmaceutical composition according to claim 9.

38. A method of inhibiting or reducing the likelihood of an occurrence of a secondary disease state or condition of HCV comprising administering to a patient at risk of a secondary disease state or condition an effective amount of a pharmaceutical composition according to claim 9.

39. The method according to claim 38 wherein said secondary disease state or condition is cirrhosis of the liver, AIDS, cancer, cryoglobulinemia, lichen planus, porphyria cutanea tarda, diabetes type II, decrease in production of clotting factors of platelet formation or Raynaud's disease

40. The method of claim 39 wherein said cancer is B cell lymphoma or hepatocellular cancer.

41. The method of claim 39 wherein said cancer is hepatocellular cancer.

42. The method of claim 39 wherein said disease state or condition is cirrhosis of the liver.
